(19) United States
(12) Patent Application Publication
(10) Pub. No.: US 2023/0105013 A1
(43) Pub. Date: Apr. 6, 2023

(54) Isolated or Engineered Polypeptides, Microorganisms as well as Method for Synthesizing Phenolic Phytochemical Phosphate Derivatives using the Polypeptides or Microorganisms

(71) Applicant: National Taiwan University, Taipei (TW)

(72) Inventors: NAN-WEI SU, Taipei (TW); CHEN HSU, Taipei (TW)

(21) Appl. No.: 17/852,836

(22) Filed: Jun. 29, 2022

(30) Foreign Application Priority Data
Oct. 1, 2021 (TW) 110136667

(51) Int. Cl. C12N 9/12; C12P 9/00; C12P 17/18; C12P 17/06

(52) U.S. Cl. CPC C12N 9/1294; C12P 9/00; C12P 17/181; C12P 17/06

(57) ABSTRACT

An isolated or engineered polypeptide, a microorganism comprising a nucleic acid sequence encoded by the polypeptide, and a method for synthesizing a polyphenolic phytochemicals phosphate derivative using the polypeptide or the microorganism are provided. The polypeptide having a homologous protein sequence that is more than 70% identical to the polyphenol phosphorylation synthetase (SEQ ID NO: 13) comprises a conserved domain which sequentially comprises: an ATP-binding domain, which includes active catalytic sites of Lys27, Arg102, and Glu282; a substrate-binding domain, which includes a conserved motif of DDHHFYIDAMLDAKAR (SEQ ID NO: 14), and includes active catalytic sites ofAsp627, His629, and His630; and a phosphorylated histidine catalytic domain, which includes His795 based on SEQ ID NO: 13.

### BACKGROUND OF THE INVENTION

### 1. Technical Field

The present invention relates to an isolated or engineered polypeptide, a microorganism comprising a nucleic acid sequence encoding the polypeptide, and a method for synthesizing a polyphenolic phytochemicals phosphate derivative using the polypeptide or the microorganism. More specifically, said polypeptide has a homologous protein sequence that is more than 70% identical to the polyphenol phosphorylation synthetase (SEQ ID NO: 13).

### 2. Description of Related Art

Many researches have shown that consumption of polyphenolic phytochemicals helps prevent or delay the occurrence of multimorbidity. It was pointed out in recent studies, however, that polyphenolic phytochemicals are absorbed to a very limited extent. For higher bioavailability, polyphenolic phytochemicals may have their physical and chemical properties improved by structural modification. Currently, the main modification reactions studied in relation to biotransformation of flavonoids are hydroxylation, dehydroxylation, O-methylation, O-demethylation, glycosylation, deglycosylation, hydrogenation, dehydrogenation, C-ring cleavage of the benzo-γ-pyrone system, cyclization, and carbonyl reduction.

Cunninghamella, Penicillium, and Aspergillus are the most commonly used microbes in cultures for transformation purposes, in particular Aspergillus niger, which can transform flavanone into flavan-4-ol, 2′-hydroxydihydrochalcone, 3-hydroxyflavone, 6-hydroxyflavanone, 4′-hydroxyflavanone, and so on.

Microbes that were used for biotransformation in previous studies also include Bacillus subtilis var. natto, which typically performs deglycosylation on glycosylated flavonoids, and Bacillus subtilis NTU-18, which was found to have high (3-glucosidase activity and be capable of highly efficient deglycosylation of glycosylated flavonoids. Moreover, research results have shown that during solid-state fermentation of black beans with Bacillus subtilis BCRC 14715, the glycosylated flavonoid content decreases whereas the deglycosylated flavonoid content increases, and that the resulting black bean natto has a higher percentage of deglycosylated flavonoids out of total flavonoids than the original black beans.

In addition, there have been researches in which Bacillus subtilis BCRC 80517 is obtained from commercially available natto products and then used to carry out phosphorylation-based modification on daidzein and genistein in order to produce daidzein 7-O-phosphate (D7P) and genistein 7-O-phosphate (G7P). D7P and G7P are a hundred thousand times as water-soluble as daidzein and genistein, respectively.

### BRIEF SUMMARY OF THE INVENTION

While there have been studies on biotransformation of flavonoids, no literature can be found that relates to modification of polyphenolic phytochemicals by microbial phosphorylation. When the structure of a polyphenolic phytochemical is modified by microbial phosphorylation, the modification conditions are milder than those of chemical modification, and there is no need to use large amounts of chemicals or organic solvents, meaning microbial phosphorylation is a relatively environmentally friendly method for structural modification. The polyphenol phosphorylation phenomenon in the present invention is a novel mode of biotransformation

In view of this, a first aspect of the present invention relates to an isolated or engineered polypeptide comprising a homologous protein sequence that is more than 70% identical to the polyphenol phosphorylation synthetase (SEQ ID NO: 13); wherein said polypeptide comprises a conserved domain which is based on the polyphenol phosphorylation synthetase (SEQ ID NO: 13) and sequentially comprises: an ATP-binding domain comprising active catalytic sites of Lys27, Arg102, and Glu282; a substrate-binding domain comprising a conserved motif of DDHHFYIDAMLDAKAR (SEQ ID NO: 14), and comprising active catalytic sites of Asp627, His629, and His630; and a phosphorylated histidine catalytic domain comprising His795.

Preferably, the polypeptide catalyzes a substrate to its phosphate derivative, and said substrate is a polyphenolic phytochemical.

Preferably, the substrate is selected from the group consisting of the following formulas:

### Ar1-L-Ar2 (I),

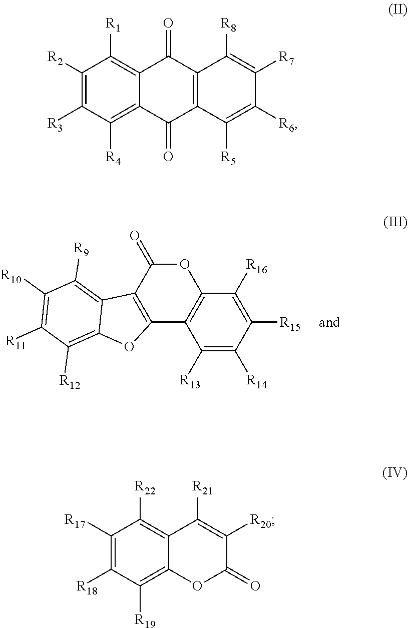

wherein Ar1 is an aryl group of the following formula:

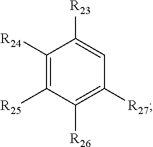

Ar2 is an aryl group of the following formula:

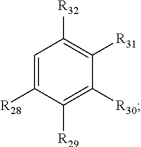

L is a linking group comprising 3 to 7 backbone carbon atoms forming a chain linking Ar1 and Ar2 as the case may be, wherein L comprises at least one of a double bond, a carbonyl group and a hydroxyl group;
R₁ to R₈ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₁ to R₈ is a hydroxyl group;
R₉ to R₁₆ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₉ to R₁₆ is a hydroxyl group;
R₁₇ to R₂₂ are respectively H, methoxy group or hydroxyl group, and at least one of R₁₇ to R₂₂ is a hydroxyl group, or R₂₀ and R₂₁, R₁₇ and R₁₈, R₁₇ and R₂₂, R₁₈ and R₁₉ or their combination are fused to form a (C₃-C₆)cycloalkyl group with hydroxyl group or a (C₆-C₁₀)aryl group with hydroxyl group;
R₂₃ to R₂₇ are respectively H, methoxy group or hydroxyl group, and at least one of R₂₃ to R₂₇ is a hydroxyl group;
R₂₈ to R₃₂ are respectively H, methoxy group or hydroxyl group, and at least one of R₂₈ to R₃₂ is a hydroxyl group;
R₃₃ to R₃₄ are respectively (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₅ is (C₁-C₅)alkyl group, (C₁-C₅)haloalkoxy group, (C₆-C₁₁)phenyl group or (C₇-C₁₂)benzyl group;
R₃₆ is (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₇ to R₄₀ are respectively (C₁-C₅)alkyl group or (C₁-C₅)haloalkoxy group; R₄₁ and R₄₂ are respectively H, (C₁-C₅)alkyl group or (C₁-C₅)haloalkoxy group, one of which is H and the other is not H;
R₄₃ and R₄₄ are respectively H, (C₁-C₅)alkyl group or (C₁-C₅)haloalkoxy group.

Preferably, the substrate is a natural or modified curcuminoid, anthraquinones, chalcone, stilbenoid, coumestan or coumarin.

Preferably, the curcuminoid is a natural or modified curcumin, bisdemethoxycurcumin, tetrahydrocurcumin or octahydrocurcumin; the anthraquinone is a natural or modified emodin, obtusifolin or aloe-emodin; the chalcone is a natural or modified phloretin, isoliquiritigenin or flavokawain A; the stilbenoid is a natural or modified resveratrol, pterostilbene, piceatannol or oxyresveratrol; the coumestan is a natural or modified coumestrol, wedelolactone or demethylwedelolactone; the coumarin is a natural or modified umbelliferone or 4-hydroxycoumarin.

Another aspect of the present invention relates to a microorganism comprising a nucleic acid sequence encoding said polypeptide.

Preferably, the nucleic acid sequence is derived from Bacillus subtilis, Bacillus halotolerans, Bacillus atrophaeus, Bacillus mojavensis, Bacillus velezensis or Bacillus amyloliquefaciens.

Another aspect of the present invention relates to a method for synthesizing a polyphenolic phytochemicals phosphate derivative, comprising: exposing a polyphenolic phytochemical to said polypeptide or to a microorganism comprising a nucleic acid sequence encoding said polypeptide for converting the polyphenol phytochemical to its phosphate derivatives.

Preferably, the polyphenolic phytochemical is a natural or modified curcuminoid, anthraquinone, chalcone, stilbenoid, coumestan or coumarin.

As stated above, the present invention ascertains important catalytic sites of a polypeptide so that when the polypeptide or a microbe having the nucleic acid sequence coding for the polypeptide is used to synthesize a polyphenolic phytochemical phosphate, the success rate of polyphenolic phytochemical phosphorylation or the yield of the synthetic polyphenolic phytochemical phosphate derivative will be increased in comparison with that of the prior art.

### BRIEF DESCRIPTION OF THE SEVERAL VIEWS OF THE DRAWINGS

The embodiments of the present invention are described by way of example only, with reference to the accompanying drawings, wherein:

FIG. 1 shows percent conversion rate of each polyphenol by the recombinant PPSs when using ATP as phosphate donor; the patterns of the bar graphs represent the ratio of phosphorylated products at different O-position in the total product yield of each polyphenol, and the trace products (yield<2%) are not counted; experiments were performed in triplicate, and the S.D. is shown.

FIGS. 2(A) to 2(C) show a chemical analysis spectrum of the reaction of the recombinant PPSs and curcumin (CUR): (A) the structure of the curcumin; (B) HPLC chromatogram of curcumin before reaction; (C) The HPLC-UV/Vis spectrum (430 nm) of the curcumin phosphate (CUR-P) produced after the recombinant PPSs reacted with curcumin for 1 hour at 40° C. and pH 7.8.

FIGS. 3(A) to 3(C) show a chemical analysis spectrum of the reaction of the recombinant PPSs and bisdemethoxycurcumin (BDMC): (A) the structure of the bisdemethoxycurcumin; (B) HPLC chromatogram of bisdemethoxycurcumin before reaction; (C) The HPLC-UV/Vis spectrum (430 nm) of the bisdemethoxycurcumin phosphate (BDMC-P) produced after the recombinant PPSs reacted with bisdemethoxycurcumin for 1 hour at 40° C. and pH 7.8.

FIGS. 4(A) to 4(C) show a chemical analysis spectrum of the reaction of the recombinant PPSs and tetrahydrocurcumin (THC): (A) the structure of the tetrahydrocurcumin; (B) HPLC chromatogram of tetrahydrocurcumin before reaction; (C) The HPLC-UV/Vis spectrum (280 nm) of the tetrahydrocurcumin phosphate (THC-P) produced after the recombinant PPSs reacted with tetrahydrocurcumin for 1 hour at 40° C. and pH 7.8.

FIGS. 5(A) to 5(C) show a chemical analysis spectrum of the reaction of the recombinant PPSs and octahydrocurcumin (OHC): (A) the structure of the octahydrocurcumin; (B) HPLC chromatogram of octahydrocurcumin before reaction; (C) The HPLC-UV/Vis spectrum (280 nm) of the octahydrocurcumin phosphate (OHC-P) produced after the recombinant PPSs reacted with octahydrocurcumin for 1 hour at 40° C. and pH 7.8.

FIGS. 6(A) to 6(C) show a chemical analysis spectrum of the reaction of the recombinant PPSs and emodin (EMD): (A) the structure of the emodin; (B) HPLC chromatogram of emodin before reaction; (C) The HPLC-UV/Vis spectrum (260 nm) of the emodin phosphate (EMD -P) produced after the recombinant PPSs reacted with emodin for 1 hour at 40° C. and pH 7.8.

FIGS. 7(A) to 7(C) show a chemical analysis spectrum of the reaction of the recombinant PPSs and obtusifolin (OBF): (A) the structure of the obtusifolin; (B) HPLC chromatogram of obtusifolin before reaction; (C) The HPLC-UV/Vis spectrum (260 nm) of the obtusifolin phosphate (OBF-P) produced after the recombinant PPSs reacted with obtusifolin for 1 hour at 40° C. and pH 7.8.

FIGS. 8(A) to 8(C) show a chemical analysis spectrum of the reaction of the recombinant PPSs and aloe-emodin (ALE): (A) the structure of the aloe-emodin; (B) HPLC chromatogram of aloe-emodin before reaction; (C) The HPLC-UV/Vis spectrum (260 nm) of the aloe-emodin phosphate (ALE-P) produced after the recombinant PPSs reacted with aloe-emodin for 1 hour at 40° C. and pH 7.8.

FIGS. 9(A) to 9(C) show a chemical analysis spectrum of the reaction of the recombinant PPSs and phloretin (PHL): (A) the structure of the phloretin; (B) HPLC chromatogram of phloretin before reaction; (C) The HPLC-UV/Vis spectrum (310 nm) of the phloretin phosphate (PHL-P) produced after the recombinant PPSs reacted with phloretin for 1 hour at 40° C. and pH 7.8.

FIGS. 10(A) to 10(C) show a chemical analysis spectrum of the reaction of the recombinant PPSs and isoliquirtigenin (ISL): (A) the structure of the isoliquirtigenin; (B) HPLC chromatogram of isoliquirtigenin before reaction; (C) The HPLC-UV/Vis spectrum (310 nm) of the isoliquirtigenin phosphate (ISL-P) produced after the recombinant PPSs reacted with isoliquirtigenin for 1 hour at 40° C. and pH 7.8.

FIGS. 11(A) to 11(C) show a chemical analysis spectrum of the reaction of the recombinant PPSs and flavokawain A (FKA): (A) the structure of the flavokawain A; (B) HPLC chromatogram of flavokawain A before reaction; (C) The HPLC-UV/Vis spectrum (280 nm) of the flavokawain A phosphate (FKA-P) produced after the recombinant PPSs reacted with flavokawain A for 1 hour at 40° C. and pH 7.8.

FIGS. 12(A) to 12(D) show a chemical analysis spectrum of the reaction of the recombinant PPSs and resveratrol (RES): HPLC chromatogram of resveratrol before (A) and after (B) the reaction of resveratrol with the recombinant PPSs for 2 hours at 40° C., pH 7.8; (C) The HPLC spectrum of (C) the RES-3P and (D) RES-4′P standards purified from semi-preparative HPCL.

FIGS. 13(A) to 13(D) show a NMR spectra of resveratrol 3-O-phosphate: (A) the structure of resveratrol 3-O-phosphate, (B) ¹H (in DMSO-d6, 500 MHz), (C) 2D HMQC ¹H/³¹P (TPP in acetone-d6, 202 MHz for ³¹P NMR), (D)¹³C (in DMSO-d6, 125 MHz) NMR spectra of resveratrol 3-O-phosphate.

FIGS. 14(A) to 14(D) show a NMR spectra of resveratrol 4′-O-phosphate: (A) the structure of resveratrol 4′-O-phosphate, (B) ¹H (in DMSO-d6, 500 MHz), (C) 2D HMQC ¹H/³¹P (TPP in acetone-d6, 202 MHz for ³¹C NMR), (D) ¹³C (in DMSO-d6, 125 MHz) NMR spectra of resveratrol 4′-O-phosphate.

FIGS. 15(A) to 15(C) show a chemical analysis spectrum of the reaction of the recombinant PPSs and pterostilbene (PTER): (A) the structure of the pterostilbene; (B) HPLC chromatogram of pterostilbene before reaction; (C) The HPLC-UV/Vis spectrum (280 nm) of the pterostilbene phosphate (PTER-P) produced after the recombinant PPSs reacted with pterostilbene for 1 hour at 40° C. and pH 7.8.

FIGS. 16(A) to 16(C) show a chemical analysis spectrum of the reaction of the recombinant PPSs and piceatannol (PCT): (A) the structure of the piceatannol; (B) HPLC chromatogram of piceatannol before reaction; (C) The HPLC-UV/Vis spectrum (280 nm) of the piceatannol phosphates (PCT-P1, PCT-P2) produced after the recombinant PPSs reacted with piceatannol for 1 hour at 40° C. and pH 7.8.

FIGS. 17(A) to 17(C) show a chemical analysis spectrum of the reaction of the recombinant PPSs and oxyresveratrol (OXY): (A) the structure of the oxyresveratrol; (B) HPLC chromatogram of oxyresveratrol before reaction; (C) The HPLC-UV/Vis spectrum (280 nm) of the oxyresveratrol phosphates (OXY-P1, OXY-P2, OXY-P3) produced after the recombinant PPSs reacted with oxyresveratrol for 1 hour at 40° C. and pH 7.8.

FIGS. 18(A) to 18(C) show a chemical analysis spectrum of the reaction of the recombinant PPSs and coumestrol (CUM): (A) the structure of the coumestrol; (B) HPLC chromatogram of coumestrol before reaction; (C) The HPLC-UV/Vis spectrum (254 nm) of the coumestrol phosphate (CUM-P) produced after the recombinant PPSs reacted with coumestrol for 1 hour at 40° C. and pH 7.8.

FIGS. 19(A) to 19(C) show a chemical analysis spectrum of the reaction of the recombinant PPSs and wedelolactone (WDL): (A) the structure of the wedelolactone; (B) HPLC chromatogram of wedelolactone before reaction; (C) The HPLC-UV/Vis spectrum (254 nm) of the wedelolactone phosphates (WDL-P1, WDL-P2) produced after the recombinant PPSs reacted with wedelolactone for 1 hour at 40° C. and pH 7.8.

FIGS. 20(A) to 20(C) show a chemical analysis spectrum of the reaction of the recombinant PPSs and demethylwedelolactone (DMWDL): (A) the structure of the demethylwedelolactone; (B) HPLC chromatogram of demethylwedelolactone before reaction; (C) The HPLC-UV/Vis spectrum (254 nm) of the demethylwedelolactone phosphates (DMWDL-P1, DMWDL-P2) produced after the recombinant PPSs reacted with demethylwedelolactone for 1 hour at 40° C. and pH 7.8.

FIGS. 21(A) to 21(C) show a chemical analysis spectrum of the reaction of the recombinant PPSs and umbelliferone (UMB): (A) the structure of the umbelliferone; (B) HPLC chromatogram of umbelliferone before reaction; (C) The HPLC-UV/Vis spectrum (320 nm) of the umbelliferone phosphate (UMB-P) produced after the recombinant PPSs reacted with umbelliferone for 1 hour at 40° C. and pH 7.8.

FIGS. 22(A) to 22(C) show a chemical analysis spectrum of the reaction of the recombinant PPSs and 4-hydroxycoumarin (4HC): (A) the structure of the 4-hydroxycoumarin; (B) HPLC chromatogram of 4-hydroxycoumarin before reaction; (C) The HPLC-UV/Vis spectrum (270 nm) of the 4-hydroxycoumarin phosphate (4HC-P) produced after the recombinant PPSs reacted with 4-hydroxycoumarin for 1 hour at 40° C. and pH 7.8.

FIGS. 23(A) to 23(M) show multiple sequence alignments of BsPPS with orthologs from different Bacillus species.

FIG. 24 shows a phylogenetic tree of PPS homologous protein sequences.

FIG. 25 shows a simulated tertiary structure for predicting PPS using LmRPH (PDB entry, 5FBU) as a template.

FIG. 26 shows a simulated structure of the substrate-binding region of PPS.

FIGS. 27(A) and (27(B) show: (A) Active sites of PPS homology model that dock with Mg2+/ATP; (B) PPSs homology model and catalytic sites of phosphorylation active region thereof that dock with substrates.

FIG. 28 shows important catalytic sites predicted by the outward extension of the PPS phosphorylation active region.

FIG. 29 shows an analysis of the protein purified by Histrap™ HP column by SDS-PAGE (M: protein molecular weight; PPS eluate (buffer solution containing 20 mM imidazole)).

FIG. 30 shows the relative specific activity of the PPS protein mutant strains.

FIG. 31 shows a far-UV circular dichroism spectrum of the PPS protein of interest and its mutant strain; the presented curve is smoothed by nonlinear regression.

### DETAILED DESCRIPTION OF THE INVENTION

The detailed description and technical content of the present invention are now described as follows in conjunction with the drawings. Furthermore, the drawings in the present invention are not necessarily drawn according to the actual scale for the convenience of description. These drawings and their scales are not intended to limit the scope of the present invention.

Unless otherwise defined, all technical and scientific terms used herein have the same meaning as commonly understood by one of ordinary skill in the art to which this invention belongs. The following terms used throughout this application shall have the following meanings.

“Or” means “and/or” unless stated otherwise. “Comprising” means not excluding the presence or addition of one or more other components, steps, operations or elements to the described component, step, operation or element, respectively. The terms “comprising”, “including”, “containing”, and “having” as used herein are interchangeable and not limiting. As used herein and in the appended claims, the singular forms “a” and “the” include plural referents unless the context otherwise dictates. For example, the terms “a”, “the”, “one or more” and “at least one” are used interchangeably herein.

The term “gene” means a genetic unit that occupies a specific locus on a chromosome and consists of transcriptional and/or translational regulatory sequences and/or coding regions and/or non-translated sequences (i.e., introns, 5′ and 3′ non-translated sequence). References to “mutations” or “deletions” in relation to a gene generally refer to changes or alterations in a gene that result in reduced or no expression of the encoded gene product or render the gene product non-functional or reduced in function compared to a wild-type gene product. Examples of such alterations include nucleotide substitutions, deletions or additions, in whole or in part, to the coding or regulatory sequences of the target gene that disrupt, eliminate, down-regulate or significantly reduce the performance of the polypeptide encoded by the gene, whether at the transcriptional or translational level. In certain aspects, the targeted gene can be rendered “non-functional” by changes or mutations at the nucleotide level that alter the amino acid sequence of the encoded polypeptide, allowing the modified polypeptide to behave, but have reduced function or activity relative to one or more enzymatic activities, whether by altering the active site of the polypeptide, its cellular localization, its stability, or other functional characteristics as will be apparent to those skilled in the art.

The terms “polypeptide,” “polypeptide fragment,” “peptide,” and “protein” are used interchangeably herein to refer to polymers of amino acid residues, and variants and synthetic analogs thereof. Accordingly, these terms apply to amino acid polymers in which one or more amino acid residues are synthetic non-naturally occurring amino acids, such as chemical analogs corresponding to naturally occurring amino acids. In certain aspects, polypeptides can include enzymatic polypeptides (or “enzymes”), which typically catalyze (i.e. increasing the rate of) various chemical reactions.

“Isolated”, as used herein, means that have been (1) separated (whether in nature and/or in an experimental context) from at least some of the components with which they were originally produced, and/or (2) engineered, produced, prepared and/or manufactured by humans. In some embodiments, a substance is “pure” if it is substantially free of other components. In some embodiments, as understood by those skilled in the art, a substance may still be considered “isolated” or even “pure” after being combined with certain other components such as one or more carriers or excipients (e.g. buffers, solvents, water, etc.); in such embodiments, the isolation or purity percentage of the material is calculated without the inclusion of such carriers or excipients. By way of example only, in some embodiments, a biopolymer such as a polypeptide or polynucleotide that occurs in nature is considered “isolated” when a) its source or origin is not the same as that found in nature in its native state associated with some or all of the components accompanied; b) it is substantially free of other polypeptides or nucleic acids from the same species as the species in which it is produced in nature; or c) expressed by cells of a species not in which it is produced in nature or other expression system or otherwise associated with components from said cell or other expression system. Thus, for example, in some embodiments, a polypeptide that is chemically synthesized or synthesized in a cell system different from the cell in which it is produced in nature is considered an “isolated” polypeptide. Alternatively or additionally, in some embodiments, a polypeptide that has been subjected to one or more purification techniques may be considered an “isolated” polypeptide to the extent that it has been separated from other components that: a) are associated in nature and/or b) are associated with which it was originally produced.

“Conserved”, as used herein, refers to the situation in biology that is similar or identical within a nucleic acid sequence, protein sequence, protein structure or polysaccharide sequence; it may occur between species, or between different molecules arising from the same organism. From an evolutionary point of view, it means a state in which a particular sequence continues to be preserved during the process of speciation.

The term “modified” does not necessarily mean that a nucleotide/amino acid analog or a non-natural nucleotide/amino acid is obtained by directly altering the natural nucleotides/amino acids, but that nucleotide/amino acid analogs or non-natural nucleotides/amino acids differ from natural nucleotides/amino acids. In some embodiments, the modification comprises chemical modification; when the embodiment involves genetic modification of microorganisms, typical strain development and/or molecular genetic techniques can be used to achieve the effect.

The present invention relates to an isolated or engineered polypeptide and a microorganism containing said polypeptide, the polypeptide comprises a homologous protein sequence that is more than 70% identical to the polyphenol phosphorylation synthetase (SEQ ID NO: 13); wherein said polypeptide comprises a conserved domain which is based on the polyphenol phosphorylation synthetase (SEQ ID NO: 13) and sequentially comprises: an ATP-binding domain comprising active catalytic sites of Lys27, Arg102, and Glu282; a substrate-binding domain comprising a conserved motif of DDHHFYIDAMLDAKAR (SEQ ID NO: 14), and comprising active catalytic sites of Asp627, His629, and His630; and a phosphorylated histidine catalytic domain comprising His795.

According to some embodiments of the present invention, the degree of identity between the amino acid sequence of the substrate binding domain and the amino acid sequence of SEQ ID NO: 1 is more than 60%; preferably at least 75%, more preferably at least 80%, even more preferably at least 85%; for example, the degree of identity is preferably more than 70%, more than 71%, more than 72%, more than 73%, more than 74%, more than 75%, More than 76%, more than 77%, more than 78%, more than 79%, more than 80%, more than 85%, more than 90%, more than 95% or more than 99%, etc. As used herein, “identity” of an amino acid sequence refers to the degree to which two sequences are mutually indistinguishable, and “similarity” refers to the same ratio and/or retention ratio between the two sequences. Those of ordinary skill in the art to which the present invention pertains should understand that the long-chain amino acids of polypeptides and proteins are only partially functional in their amino acid sequences, which are called functional motifs. Proteins have the same function when they have the same functional motif; in general, when the amino acid sequence of a polypeptide or protein is at least 40% identical, it has the same function (refer to How Proteins Work, Williamson, 2011). After alignment with homologous proteins or polypeptides, the amino acid sequence of the substrate-binding domain can be at least 60% identical to SEQ ID NO: 1, preferably at least 75%, more preferably at least 80%, even more preferably at least 85% identical amino acid sequences can have the same function.

A comparison between homologous proteins or polypeptides reveals that the amino acid sequences of the ATP-binding domain and of the phosphorylated histidine catalytic domain are relatively conserved in general and are not prone to much variation. The amino acid sequence of the ATP-binding domain is preferably SEQ ID NO: 2, and the amino acid sequence of the phosphorylated histidine catalytic domain is preferably SEQ ID NO: 3.

The polyphenolic phytochemical phosphate synthetase (PPS) of interest in the present invention has 839 amino acids (SEQ ID NO: 13), a molecular weight of 94.9 kDa, a pI value of 4.81, a Mowse score of 765, a protein sequence coverage of 31%, and a unique peptide sequence identified by protein mass spectrometry, with the gene of the target protein being a predicted protein gene (gene ID: 14103593) composed of 2520 bases. The nucleic acid sequence of the isolated, purified protein is known by way of gene cloning and DNA sequencing. More specifically, the nucleic acid sequences of those amino acid sequences capable of generating the substrate binding domain, the ATP binding domain, and the phosphorylated histidine catalytic domain are SEQ ID NO:4, SEQ ID NO: 5, and SEQ ID NO: 6 respectively.

The phosphorylation stated herein refers to the addition of a phosphate group to a protein or another type of molecule. This reaction plays an important role in energy metabolism and signal transduction in a living body and is a critical to biochemistry. Currently known phosphorylation entails kinase (which is a phophotransferase) or phosphorylase, both falling within the EC 2.7 category, and the reaction requires ATP as the source of energy and Mg′ ions as a cofactor. Generally, the aforesaid enzymes hydrolyze ATP and transfer γ-phosphate to the substrate. Protein kinase is the most common large-molecule phosphorylation enzyme, is responsible for modifying, through phosphorylation, a wide range of proteins with different functions, and is an essential means for regulating signal transduction in a living body. More and more physiological phenomena, such as whether an enzyme is activated or not, have been found to be related to the phosphorylation or dephosphorylation of protein. Certain amino acid sites on a protein molecule, such as the —OH functional group of serine, threonine, or tyrosine, or the imidazole ring of histidine, can be modified by protein kinase through phosphorylation such that the molecule is activated by the addition of phosphoric acid, and this phosphoric acid can be subsequently removed with protein phosphatase to render the molecule deactivated. There are also many examples in which similar reactions produce the opposite effects. As to small-molecule phosphorylation enzymes such as acetokinase, glycerokinase, arginine kinase, shikimate kinase, mevalonate kinase, and nucleoside kinase, they are responsible for such crucial catalytic reactions in the metabolic pathways in a living body as glycolysis, the biosynthesis of amino acids, the biosynthesis of cholesterol, and the biosynthesis of nucleotides. Phosphorylation enzymes can also be divided by the source of the phosphate group into the following two types. The first type uses a phosphoric acid monoester as the phosphate donor and is generally capable of hydrolyzing ATP and transferring γ-phosphate to the substrate. The second type uses a phosphonate diester or pyrophosphate as the phosphate donor instead.

Previous studies have found that B. subtilis BCRC 80517 can transfer the phosphate group to the hydroxyl group of a flavonoid, and based on its biochemical actions, this phosphorylation enzyme should be classified into the EC 2.7 category, in which, however, there are no enzymes for phosphorylating similar substrates. No research has so far been conducted on enzymes capable of phosphorylating polyphenolic phytochemicals, and there is even no published paper related to microbe-based production of polyphenolic phytochemical phosphates or enzymatically phosphorylated polyphenolic phytochemicals. This explains why the polyphenolic phytochemical phosphate synthetase obtained by purifying the bacterium body of B. subtilis BCRC 80517 in previous studies was classified into the EC 2.7.9 category after it was found by LC-MS/MS and a bioinformatic analysis that the polyphenolic phytochemical phosphate synthetase; pyruvate, phosphate dikinase (PPDK); and phosphoenolpyruvate synthase (PEPS) have partially identical functional domains, similar sequences, and hence presumably the catalysis mechanism of PEP-utilizing enzymes. Enzymes such as PEP-utilizing enzymes carry out phosphate group transfer by forming a phospho-histidine intermediate and have three conserved domains: the PEP/pyruvate-binding domain, the PEP-utilizing domain/mobile domain, and the PEP-utilizing domain/C-terminal, in which the PEP-utilizing domain/mobile domain is a catalytic domain consisting of 15-30 amino acids, can rotate freely between the other domains, and includes a highly conserved histidine residue responsible for the transfer of the phosphate group. Such enzymes have the same working mechanism. The following paragraphs describe the reaction processes of certain PEP-utilizing enzymes, namely pyruvate, phosphate dikinase (PPDK); phosphoenolpyruvate synthase (PEPS); and rifampicin phosphotransferase (RPH).

PPDK (EC 2.7.9.1) can catalyze the reaction: ATP+pyruvate+Pi↔AMP+PEP+PPᵢ, which is a reversible reaction. PPDK can be divided into three domains, which are the PPDK_N, PEP-utilizers, and PEP-utilizers_C domains. ATP (adenosine triphosphate) binds to the PPDK_N domain at the N terminal to begin with and is thus hydrolyzed into AMP (adenosine monophosphate) and the pyrophosphate group (abbreviated as PP). The PEP-utilizers domain then rotates to the N terminal such that PPᵢ is transferred to the histidine of the PEP-utilizers domain, forming a phospho-histidine (His-P_{β}P_{γ}) intermediate. Once the intermediate reacts with the phosphate group (Pᵢ), the P_{γ} segment is cut off, forming His-P_{β} and PPᵢ. The PEP-utilizers domain then rotates to the C terminal to phosphorylate the pyruvate bound to the PEP-utilizers_C domain, and PEP is formed as a result.

PEPS (EC 2.7.9.2) can catalyze the reversible reaction between ATP, pyruvate, and H₂O to produce AMP, PEP, and Pᵢ. The catalysis mechanism is the same as that of PPDK except that during the reaction process, the phospho-histidine (His-P_{β}P_{γ}) intermediate reacts with H₂O instead to cut off the P_{γ} segment and form His-P_{β} and P_{γ}. PEPS and PPDK, therefore, have the same conserved domains. When a microbe is cultured with pyruvate or lactate serving as the carbon source, PEP production is the main reaction of PEPS. This indicates that PEPS has an important role in gluconeogenesis.

RPH (EC 2.7.9.6) is an enzyme capable of catalyzing phosphorylation-based modification of the OH group at C₂₁ of rifampicin. It can catalyze the reaction between ATP, rifampicin, and H₂O to produce AMP, rifampicinphosphate, and Pᵢ. While the conserved domains of RPH are arranged in a different order from that of PPDK, the reaction mechanisms of RPH and PPDK are quite similar. The ATP-binding domain at the N terminal of the amino acid sequence of RPH functions as the PPDK_N domain and is responsible for binding with ATP. The phosphorylated histidine catalytic domain at the C terminal of the amino acid sequence of RPH is the PEP-utilizing domain/mobile domain, includes a highly conserved histidine, and has the same reaction mechanism as its counterpart in PPDK and PEPS, except that its substrate-binding domain is located in a middle portion of the sequence whereas the substrate-binding domain of PPDK is at the C terminal of the sequence.

In a previous study, the amino acid sequence of the polyphenolic phytochemical phosphate synthetase obtained was compared with that of a known PPS, and the comparison result shows that the two synthetases have the same functional domains and similar primary sequences. Subsequent experiments further indicated that the two synthetases have the same enzyme mechanism. The polyphenolic phytochemical phosphate synthetase obtained in that study was named flavonoid phosphate synthetase (FPS) and was classified into the EC 2.7.9.×¹ enzyme category. FPS can transfer two of the phosphate groups of ATP to a flavonoid and water.

In the present invention, the sequence alignment of amino acid sequence identity is obtained by any general amino acid sequence alignment method and amino acid sequence alignment tool. The amino acid alignment method is such as but not limited to: Needleman-Wunsch algorithm, Smith-Waterman algorithm, or Karlin & Altschul algorithm; and the amino acid sequence alignment tool is such as but not limited to: BLAST (Basic Local Alignment Search Tool) program, BLAT (BLAST-like Alignment Tool) program, Gapped BLAST program or FASTA program.

Regarding the catalytic sites stated herein, the inventor of the present invention has found a number of important catalytic sites through an extensive research, including Lys27, which is related to ATP binding, and His795, which is responsible for carrying and transferring the phosphate group. In some embodiments, the ATP-binding domain includes such catalytically active sites as Lys27, Arg102, and Glu282, all of which are related to ATP binding. In some other embodiments, the phosphorylated histidine catalytic domain includes His795, which is a catalytic site that carries and transfers the phosphate group. As for the important catalytic sites in the substrate-binding domain, the inventor has found that the catalytically active sites Asp627, His629, and His630 in the substrate-binding domain, as well as His795 (which is responsible for carrying and transferring the phosphate group) in the phosphorylated histidine catalytic domain, of the polypeptide in question are important active sites in the catalysis of phosphorylation. Under certain circumstances, the substrate-binding domain includes a conserved sequence whose amino acid sequence is DDHHFYIDAMLDAKAR (SEQ ID NO: 14).

The polypeptide of the present invention or microorganisms containing nucleic acid sequences encoding the polypeptide can catalyze a substrate to their phosphate derivatives. Said substrate may be a polyphenolic phytochemical, for example: the substrate is a natural or modified curcuminoid, anthraquinones, chalcone, stilbenoid, coumestan or coumarin. More specifically, the curcuminoid is a natural or modified curcumin, bisdemethoxycurcumin, tetrahydrocurcumin or octahydrocurcumin; the anthraquinone is a natural or modified emodin, obtusifolin or aloe-emodin; the chalcone is a natural or modified phloretin, isoliquiritigenin or flavokawain A; the stilbenoid is a natural or modified resveratrol, pterostilbene, piceatannol or oxyresveratrol; the coumestan is a natural or modified coumestrol, wedelolactone or demethylwedelolactone; the coumarin is a natural or modified umbelliferone or 4-hydroxycoumarin.

In terms of chemical structure, the substrate is selected from the group consisting of the following formulas:

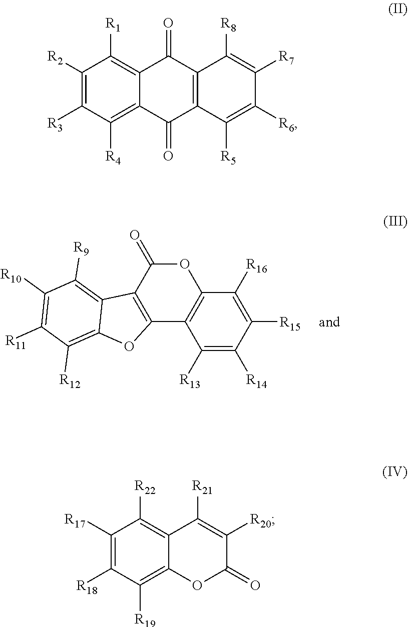

Ar1-L-Ar2 (I),

wherein Ar1 is an aryl group of the following formula:

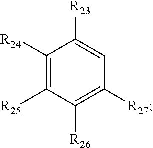

Ar2 is an aryl group of the following formula:

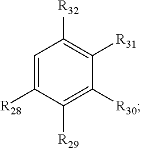

L is a linking group comprising 3 to 7 backbone carbon atoms forming a chain linking Ar1 and Ar2 as the case may be, wherein L comprises at least one of a double bond, a carbonyl group and a hydroxyl group;

R₁ to R₈ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₁ to R₈ is a hydroxyl group;

R₉ to R₁₆ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₉ to R₁₆ is a hydroxyl group;

R₁₇ to R₂₂ are respectively H, methoxy group or hydroxyl group, and at least one of R₁₇ to R₂₂ is a hydroxyl group, or R₂₀ and R₂₁, R₁₇ and R₁₈, R₁₇ and R₂₂, R₁₈ and R₁₉ or their combination are fused to form a (C₃-C₆)cycloalkyl group with hydroxyl group or a (C₆-C₁₀)aryl group with hydroxyl group;

R₂₃ to R₂₇ are respectively H, methoxy group or hydroxyl group, and at least one of R₂₃ to R₂₇ is a hydroxyl group;

R₂₈ to R₃₂ are respectively H, methoxy group or hydroxyl group, and at least one of R₂₈ to R₃₂ is a hydroxyl group;

R₃₃ to R₃₄ are respectively (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;

R₃₅ is (C₁-C₅)alkyl group, (C₁-C₅)haloalkoxy group, (C₆-C₁₁)phenyl group or (C₇-C₁₂)benzyl group;

R₃₆ is (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;

R₃₇ to R₄₀ are respectively (C₁-C₅)alkyl group or (C₁-C₅)haloalkoxy group;

R₄₁ and R₄₂ are respectively H, (C₁-C₅)alkyl group or (C₁-C₅)haloalkoxy group, one of which is H and the other is not H;

R₄₃ and R₄₄ are respectively H, (C₁-C₅)alkyl group or (C₁-C₅)haloalkoxy group.

The microorganism of the present invention comprises a nucleic acid sequence encoding the aforementioned polypeptide. The nucleic acid sequence is derived from Bacillus or Streptococcus. For example, the nucleic acid sequence is derived from Bacillus subtilis, Bacillus halotolerans, Bacillus atrophaeus, Bacillus mojavensis, Bacillus velezensis or Bacillus amyloliquefaciens. More preferably, it is derived from Bacillus subtilis. The polypeptides with the polyphenol phytochemical phosphate synthetase activity of the above strains are homologous, and after comparison with the homologous amino acid sequences of other strains, the full-length amino acid sequence of the Bacillus subtilis (SEQ ID NO: 7) is 99% identical with SEQ ID NO: 13 of the present invention; the amino acid sequence of the Bacillus halotolerans (SEQ ID NO: 8) is 89% identical to SEQ ID NO: 13 of the present invention; the amino acid sequence of the Bacillus mojavensis (SEQ ID NO: 9) is 88% identical to the SEQ ID NO: 13 of the present invention; The amino acid sequence of the Bacillus atrophaeus (SEQ ID NO: 10) is 75% identical to the SEQ ID NO: 13 of the present invention; the amino acid sequence of the Bacillus velezensis (SEQ ID NO: 11) is 71% identical to the SEQ ID: 13 of the present invention; the amino acid sequence of the Bacillus amyloliquefaciens (SEQ ID NO:12) is 70% identical to SEQ ID NO: 13 of the present invention.

The above-mentioned microorganism can normally express the nucleic acid sequence after transfer or transformation of the genetic material of the above-mentioned polypeptide (sequentially comprising the amino acid sequence of the ATP-binding domain, the substrate-binding domain and the phosphorylated histidine catalytic domain) through genetic engineering or molecular biotechnology.

The aforementioned microorganism may be a microorganism that has been genetically modified to express the aforementioned polypeptide, which may include genetic modification of the organism to enhance or enhance the production of the polypeptide in the organism. A genetically modified microorganism may be a genetically modified bacterium, unicellular organisms, microalgae, fungi, etc. This genetically modified microorganism has a genome modified (i.e. mutated or altered) from its normal form (i.e. wild-type or naturally occurring) such that the desired result can be achieved. Genetic modification of microorganisms can be accomplished using typical strain development and/or molecular genetic techniques. Such techniques are known in the art and are generally disclosed for use with microorganisms. A genetically modified microorganism may be a microorganism in which a nucleic acid molecule has been inserted, deleted, or modified (i.e. mutated; such as by nucleotide insertions, deletions, substitutions, and/or inversions) in such a way that this kind of modifications can provide the desired effect on the microorganism.

From another aspect, a method for synthesizing a polyphenolic phytochemicals phosphate derivative provided herein comprises: exposing a polyphenolic phytochemical to the aforementioned polypeptide or to a microorganism comprising a nucleic acid sequence encoding the aforementioned polypeptide for converting the polyphenol phytochemical to its phosphate derivatives.

The method of the present invention for synthesizing a polyphenolic phytochemical phosphate derivative can transform a polyphenolic phytochemical into its phosphate derivatives. Those polyphenolic phytochemical phosphate derivatives have a higher absorption rate and higher bioavailability than non-phosphorylated polyphenolic phytochemicals and, thanks to their advantageous bioactivity, can be used to make food, pharmaceuticals, industrial materials, and so on. Some examples of the aforesaid food are nutritional supplements, health food, functional food, baby food, and food for the elderly. Such food may be a solid, a fluid, a liquid, or a mixture of the above, preferably a liquid. When a pharmaceutical (e.g., a prodrug) is made, there is no special limitation on its dosage form. For example, the dosage form may be a solution, a paste, a gel, a solid, powder, or any other forms. If necessary, the pharmaceutical may include another pharmaceutically active ingredient (e.g., an anti-inflammation ingredient) or an auxiliary ingredient (e.g., a lubricating ingredient or a vehicle ingredient).

The following non-limiting embodiments of the invention are provided primarily to illustrate aspects of the invention and the benefits achieved. However, it should be understood that these examples are only for helping to make the present invention easier to understand, and are not intended to limit the scope of the present invention.

### Embodiment

Bacillus subtilis BCRC 80517 was obtained through a screening process and was deposited at the Bioresource Collection and Research Center of the Food Industry Research and Development Institute. Bacillus amyloliquefaciens BCRC 23350 and Bacillus mojavensis BCRC 17124 were purchased from the Bioresource Collection and Research Center. Any bacterial strain that required short-term storage was kept in a 4° C. solid-state nutrient agar (NA) culture medium, with continued subculturing to ensure the activity of the strain. Any bacterial strain that required long-term storage was kept at −80° C. in a 25% glycerol solution.

The gene of the polyphenolic phytochemical phosphate synthetase (PPS) for use in this embodiment was cloned separately from B. subtilis BCRC 80517, B. amyloliquefaciens BCRC 23350, and B. mojavensis BCRC 17124 such that the recombinant genes pps_Bsub::pET47b(+), pps_Bamy::pET47(+), and pps_Bmoj::pET47b(+) were formed. E. coli DH5α was used as the cloning host, and E. coli BL21 (DE3) as the expression host. The bacterial strains were kept in −80° C. 25% glycerol solutions for long-term storage.

To start with, the bacterial strains were activated, and seed culturing was subsequently carried out. IPTG was then used to induce high-level expression of the recombinant protein genes. The steps performed for each strain are as follows. 100 mL of liquid TB culture medium containing 50 μg/mL kanamycin was inoculated with 5 mL of seed. After the seed was cultured at 37° C. and 150 rpm for 2 hours, the OD600 absorbance value reached somewhere between 0.6 and 0.8. Then, 40 mL of 500 mM IPTG was added to induce expression of the recombinant protein, with the final IPTG concentration being 0.5 mM. The culturing continued at 16° C. and 100 rpm for three days, before the bacterium body was centrifugally collected (6,000 rpm, 4° C., 20 min). The collected bacterium body was washed twice with an enzyme buffer solution, and the resulting bacterial pellets were collected and stored in a −20° C. refrigerator.

The bacterium body obtained from 100 mL of the bacterial solution was re-suspended with 30 mL of cell lysis buffer solution and then vibrated until there were no noticeable clumps in the solution. After that, the solution was subjected to two ultrasonic disruption sessions in an iced water bath. Each session lasted 10 minutes (which included repeated cycles each consisting of 9 seconds of disruption and 5 seconds of rest), and there was a 5-minute rest between the two sessions. Following the disruption, low-temperature centrifugation was performed (15,000 rpm, 4° C., 30 min). The resulting supernatant was collected and filtered through a 0.22 μm PVDF membrane to become a crude enzyme extract. The crude enzyme extract was purified by FPLC, or more specifically by affinity chromatography, and an enzyme liquid was obtained as a result.

50 μL of enzyme reaction liquid (which contained a 1 mM reaction substrate (pre-dissolved in DMSO), a 50 mM Tris-HCl buffer solution, 100 mM NaCl, 10 mM ATP, and 10 mM MgCl₂, and which had a pH value of 7.8) and 50 μL of the enzyme solution (at a concentration of 100 μg/mL) were evenly mixed and allowed to react at 40° C. for 10 minutes, at the end of which 900 μL of methanol was added to terminate the reaction. After vibration-based mixing and centrifugation (13,000 rpm, 4° C., 5 minutes), the resulting supernatant was collected, diluted, and then analyzed by HPLC. The activity unit was defined as the amount of enzyme required to yield 1 nmol of product per unit time.

I. Determination of the Enzymatic Activity of the Recombinant PPSs

In this embodiment, the effects of using the PPSs obtained to catalyze the phosphorylation of a plurality of polyphenolic phytochemical substrates were further evaluated by allowing a 2 mM polyphenolic phytochemical substrate, 10 mM Mg²⁺/ATP, and 200 mL of a reaction buffer solution containing 1 mg/mL (10 μm) recombinant PPS to react for 2 hours. The activity unit was defined as the amount of enzyme required to yield 1 nmol of polyphenolic phytochemical phosphate derivative per unit time.

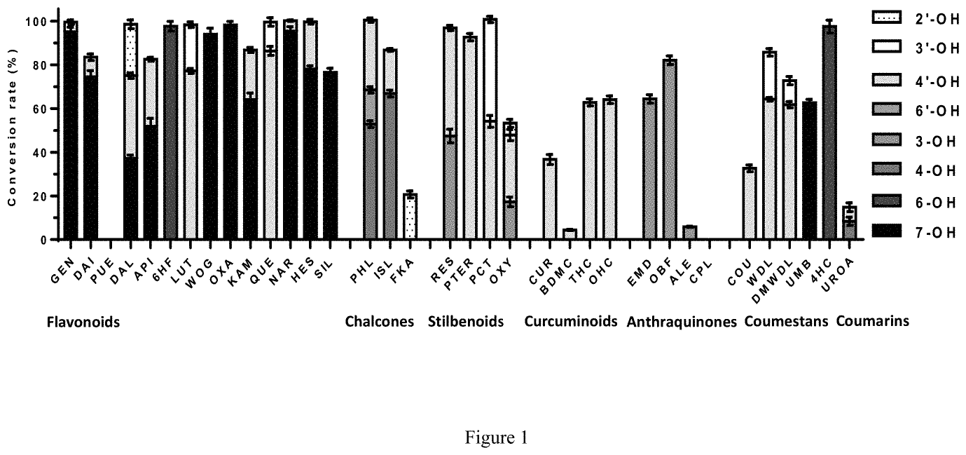

The catalyzing effects of the recombinant PPSs on each type of substrates are shown in FIG. 1, in which it can be seen that the recombinant PPSs were effective (indicated by the biotransformation rates) in phosphorylating various flavonoids, including genistein, and that the recombinant PPSs were also effective in phosphorylating such substrates as curcuminoids, anthraquinones, chalcones, stilbenes, coumestrols, and coumarins.

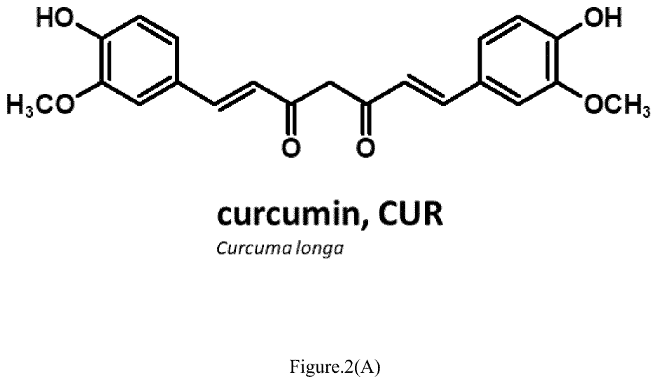

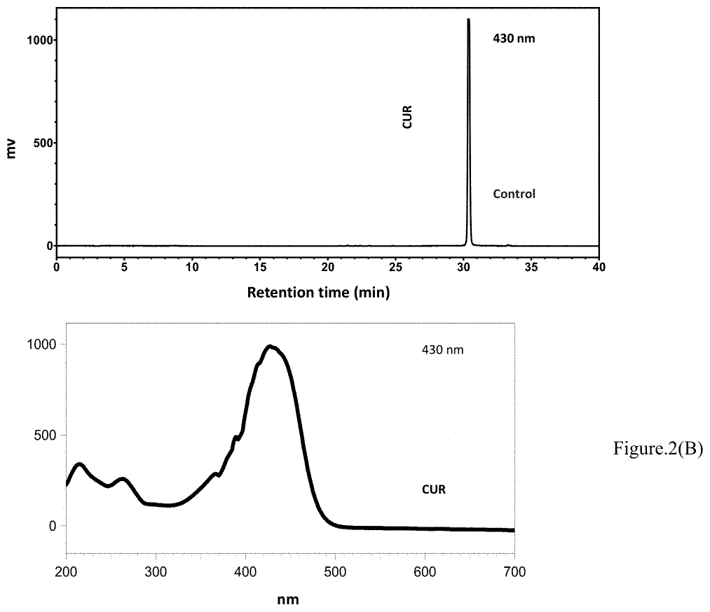

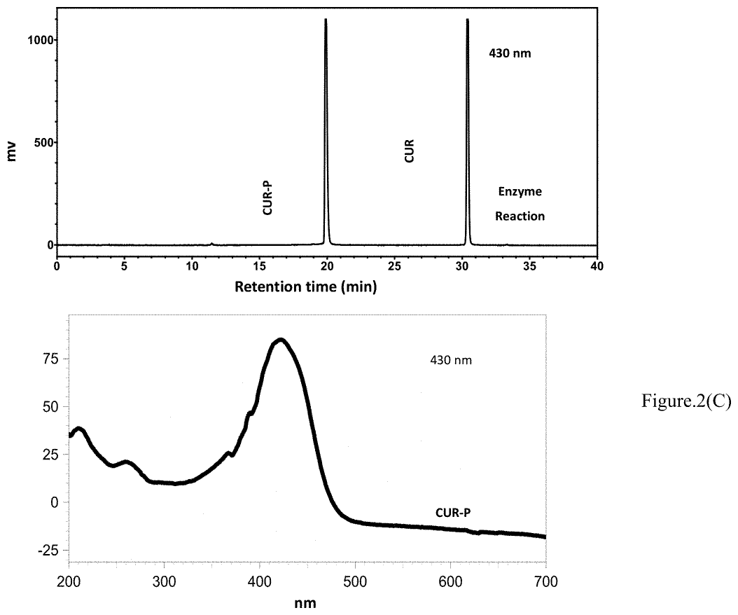

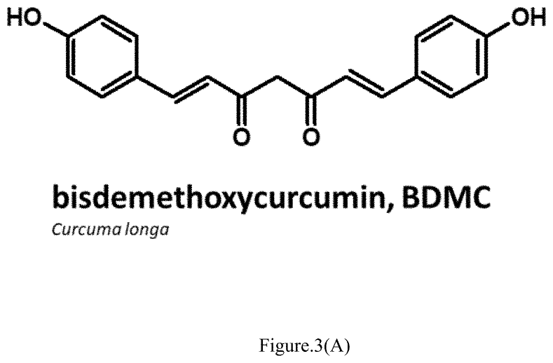

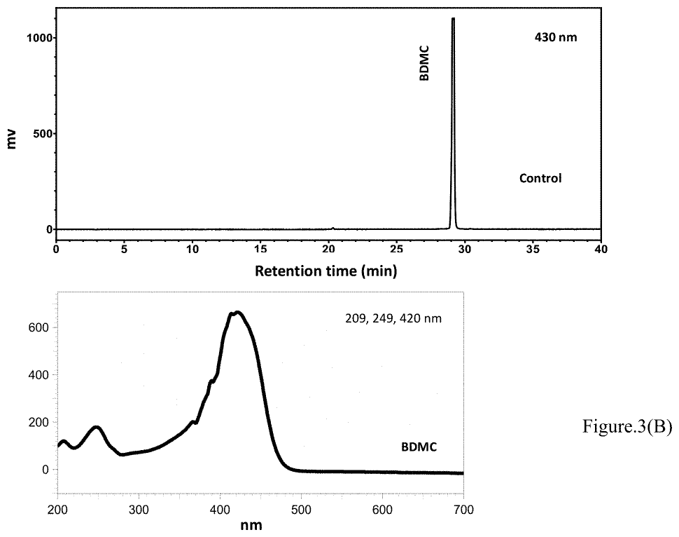

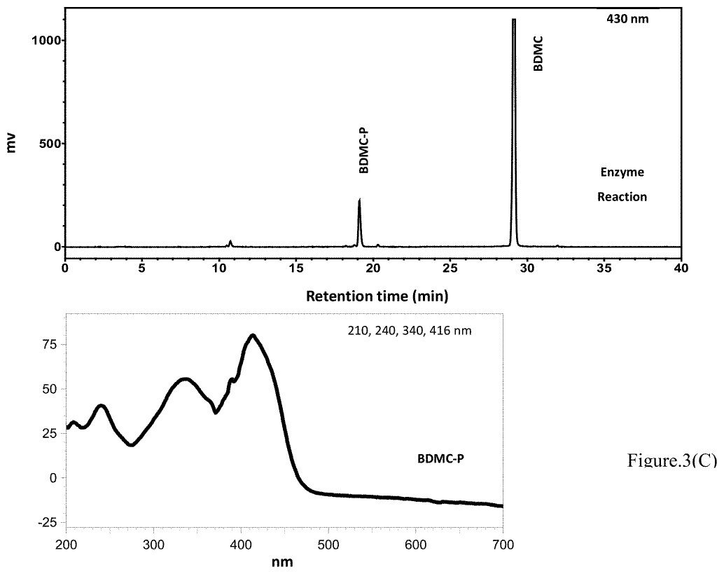

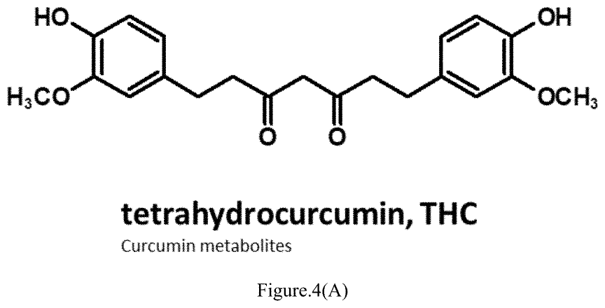

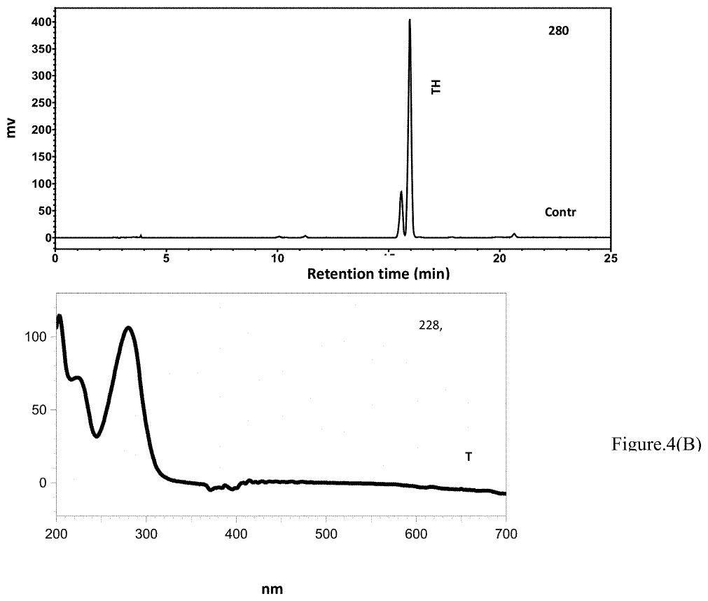

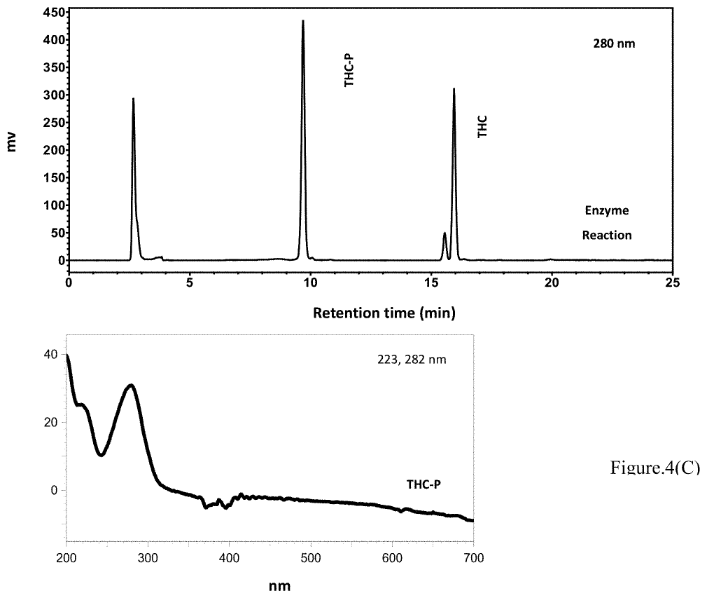

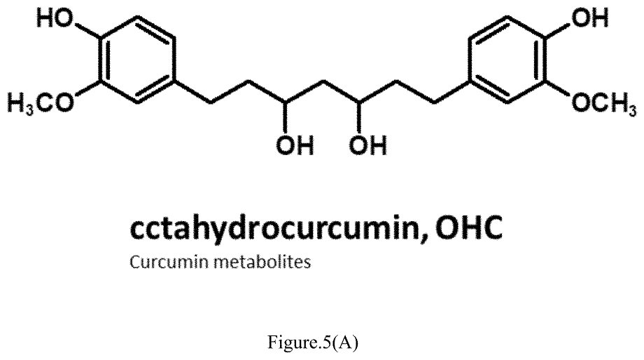

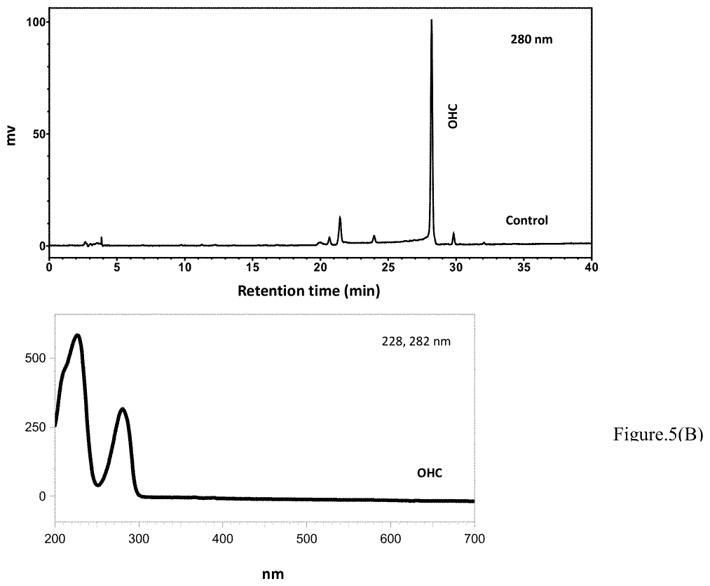

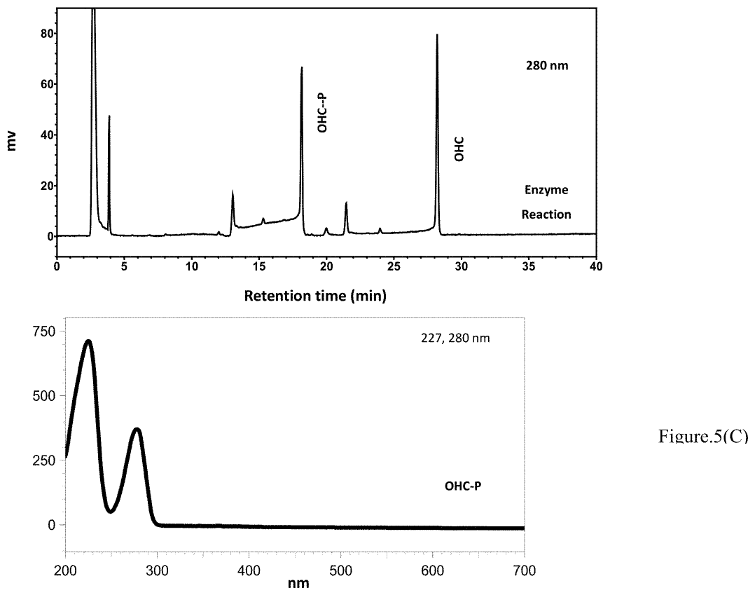

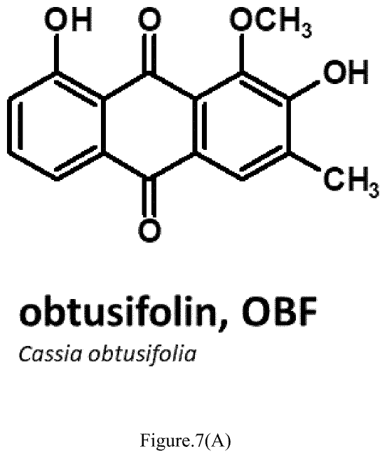

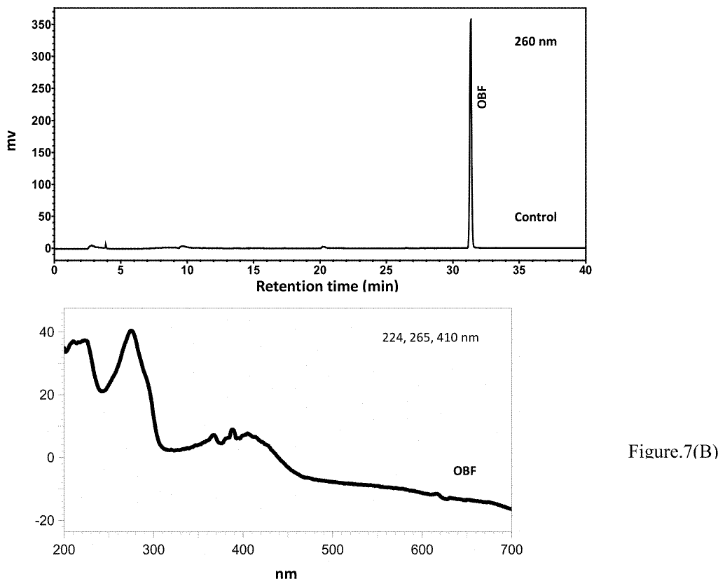

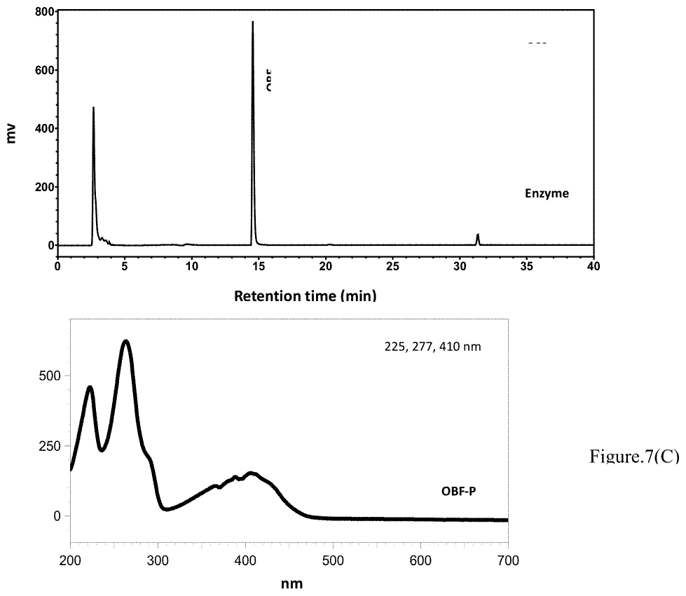

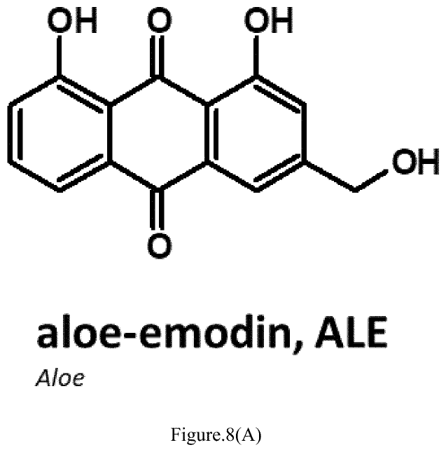

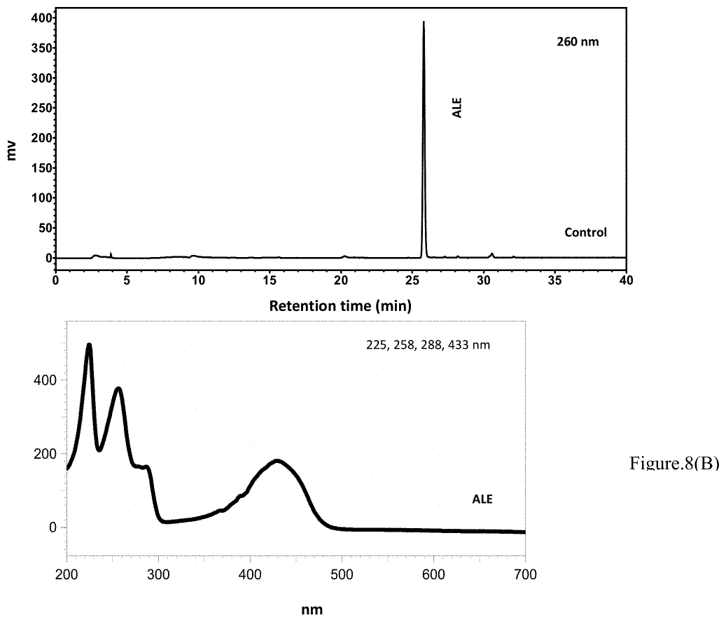

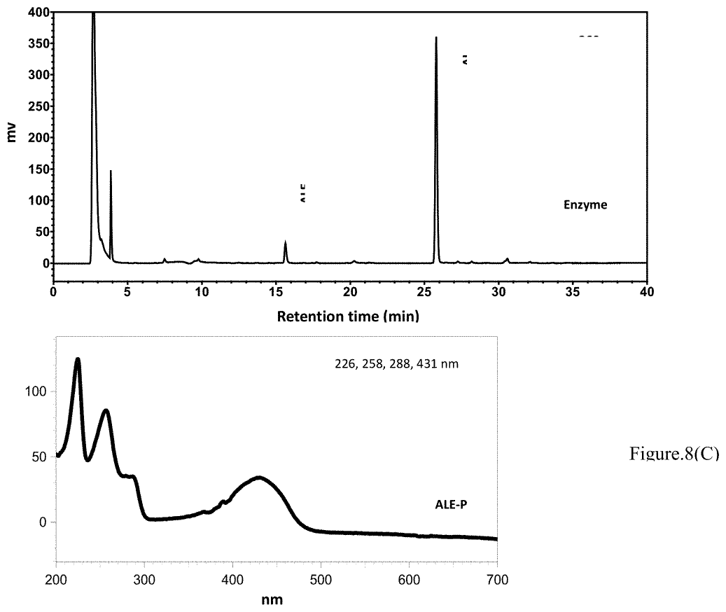

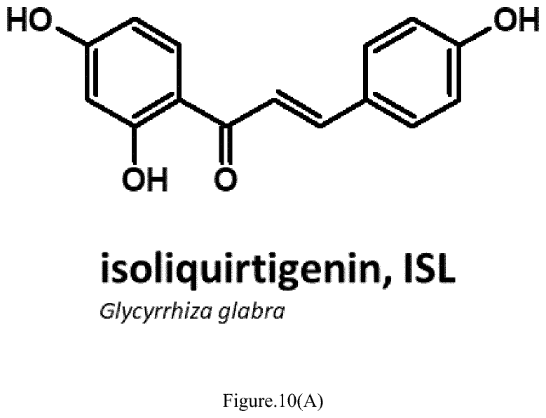

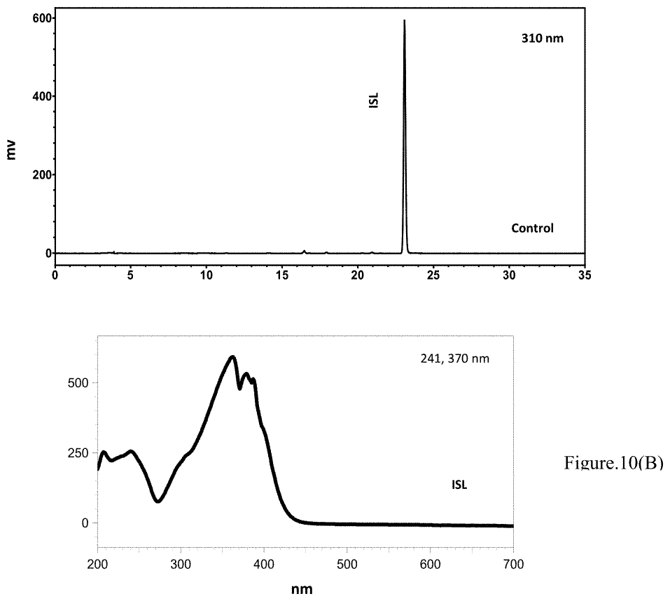

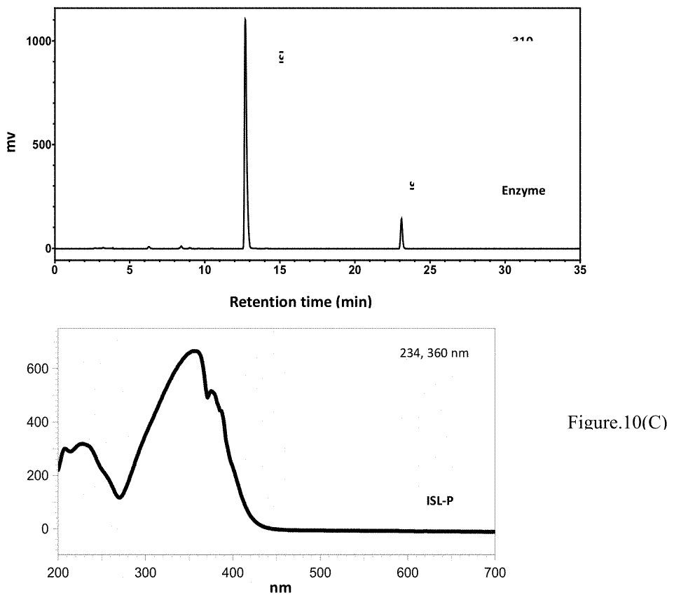

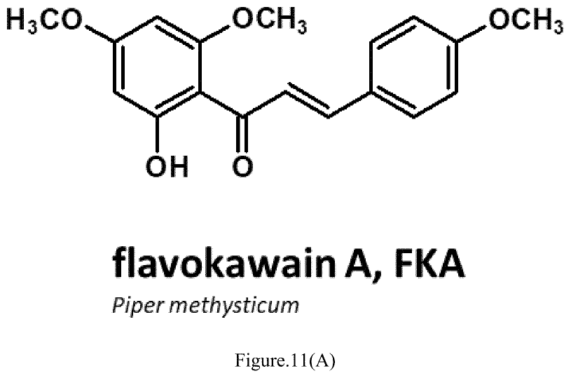

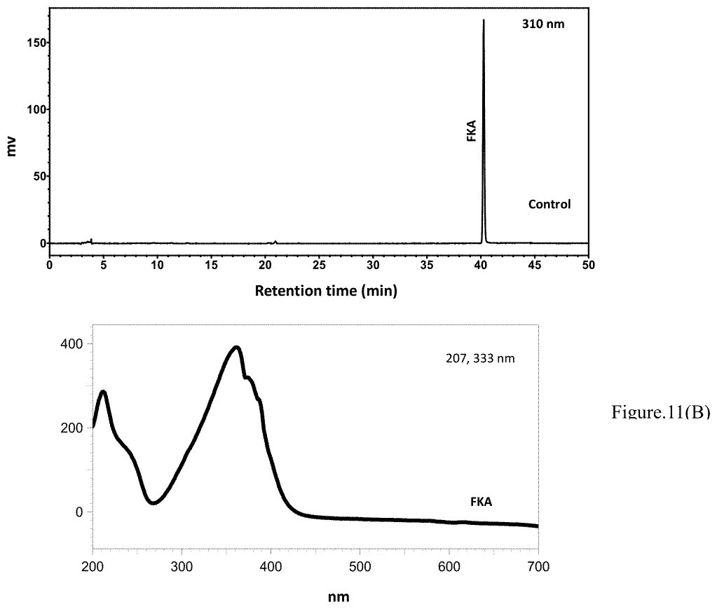

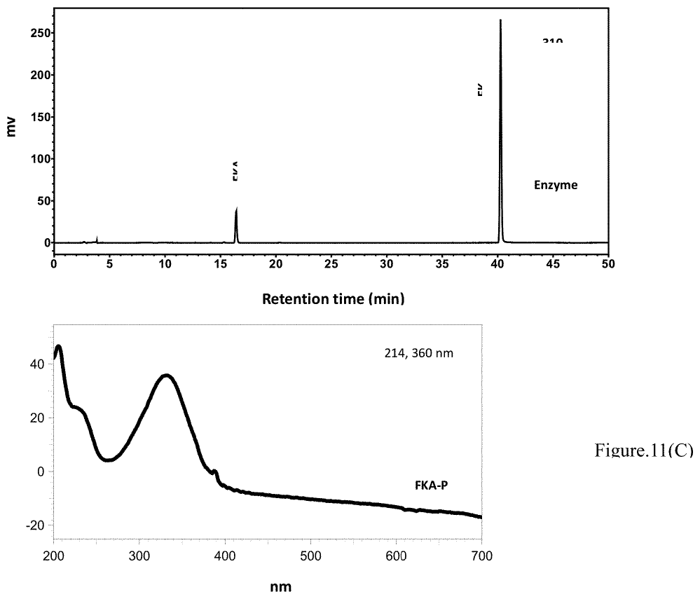

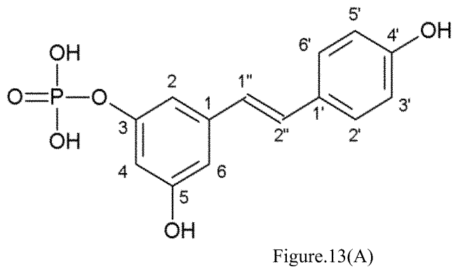

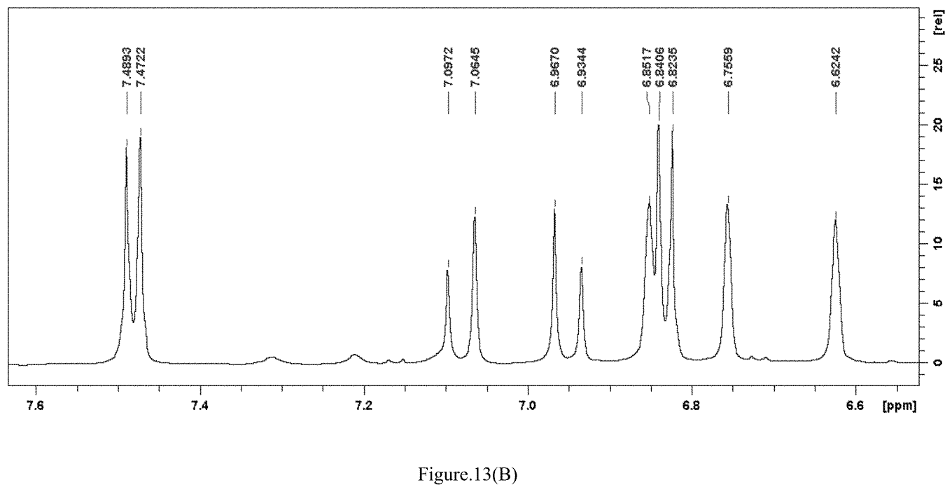

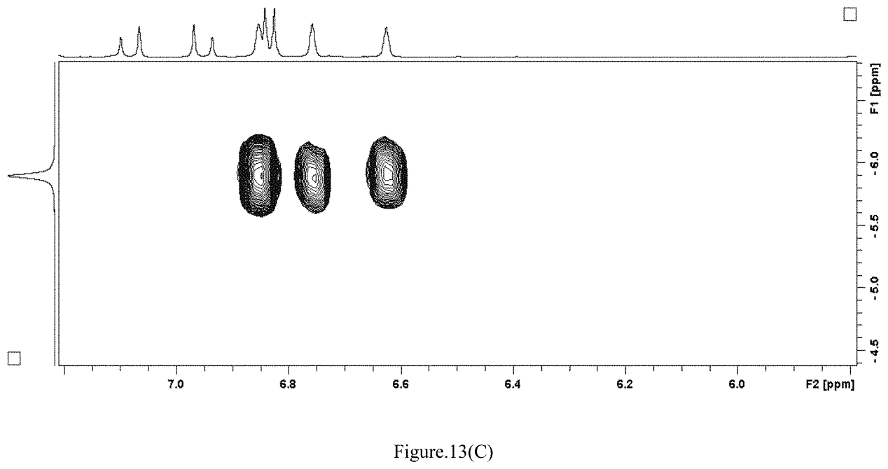

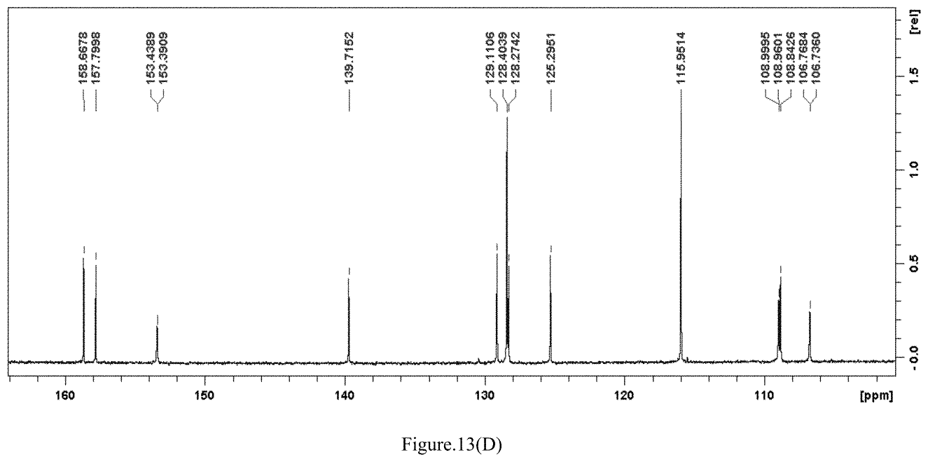

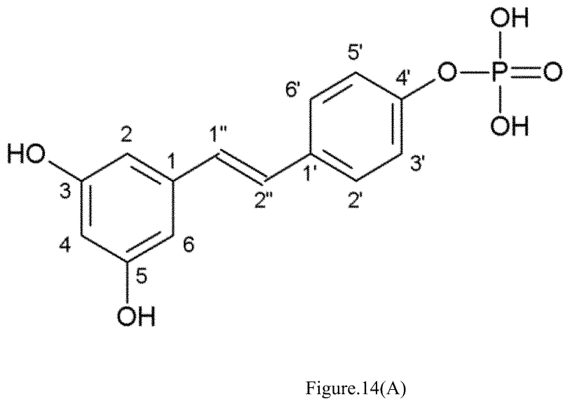

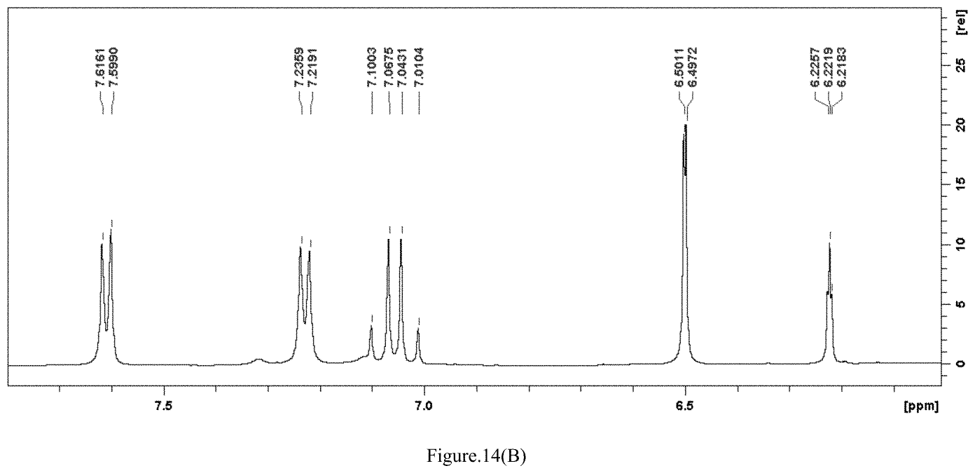

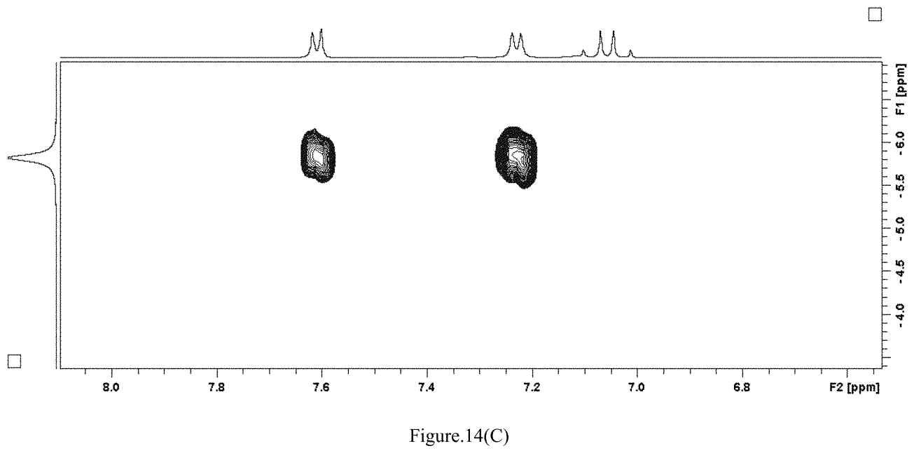

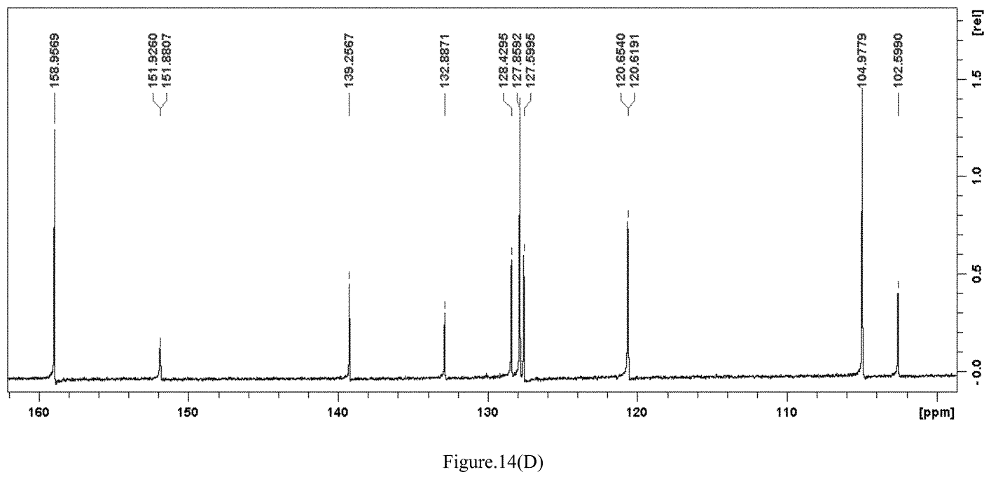

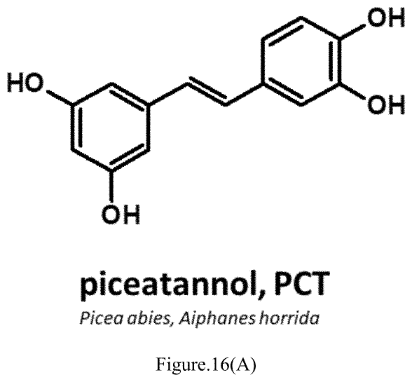

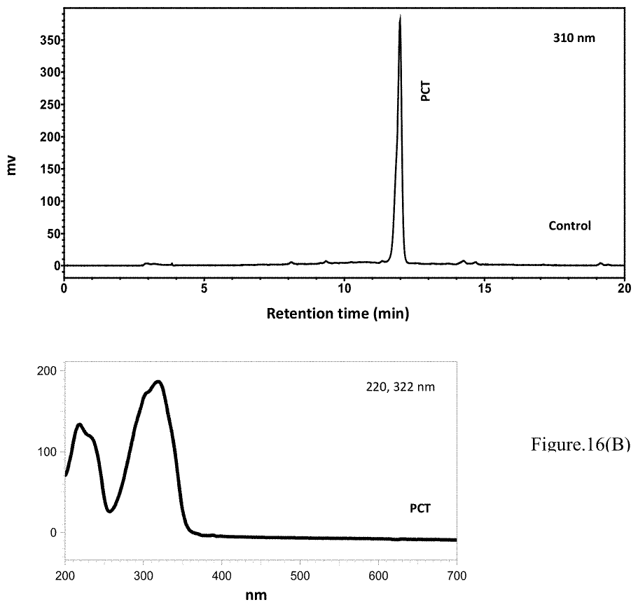

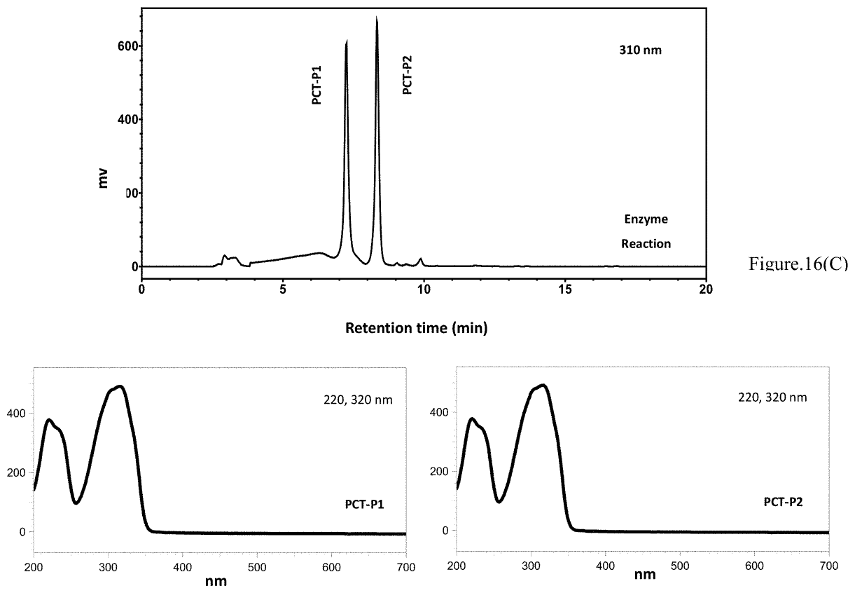

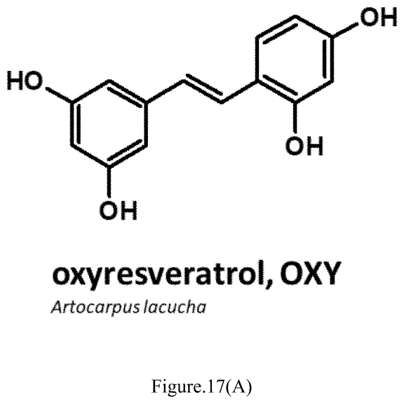

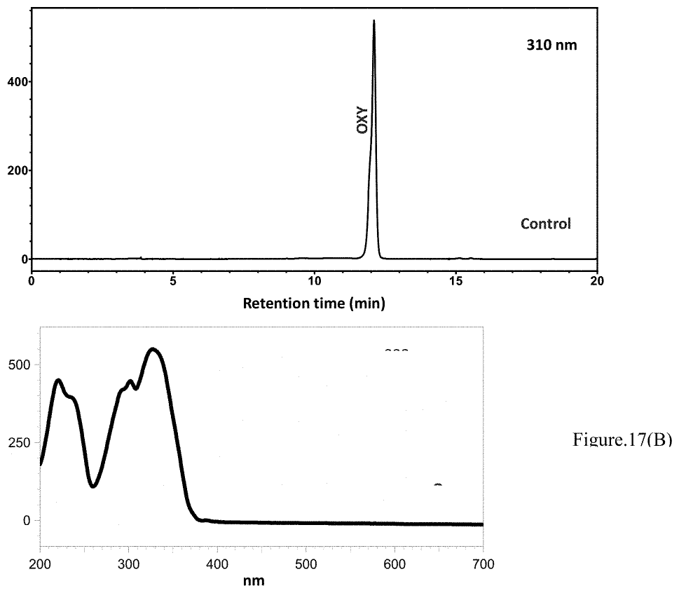

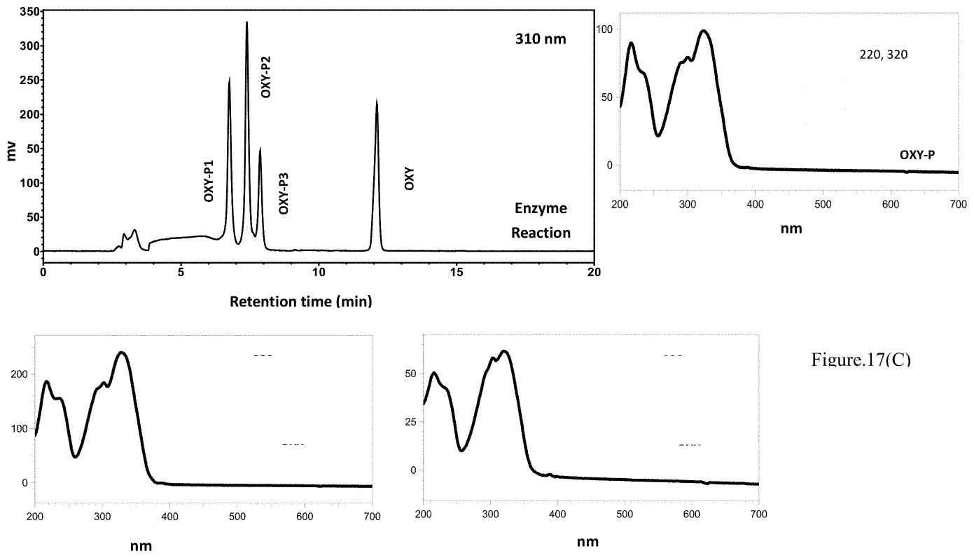

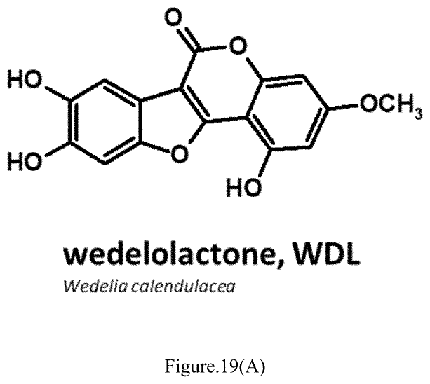

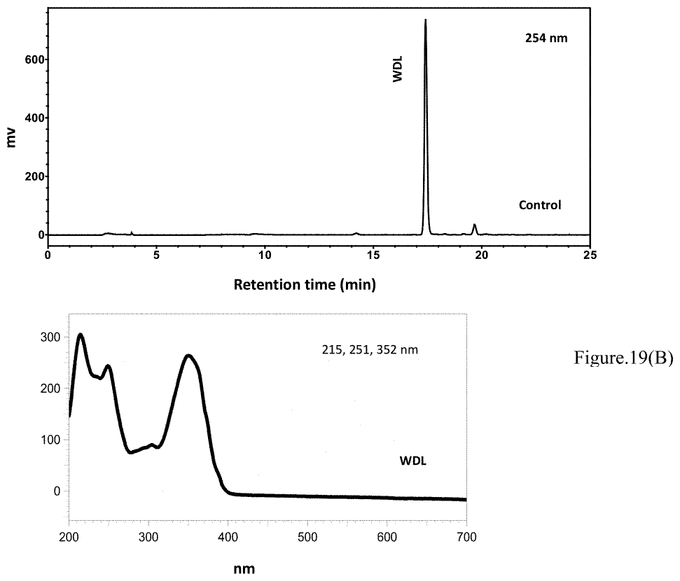

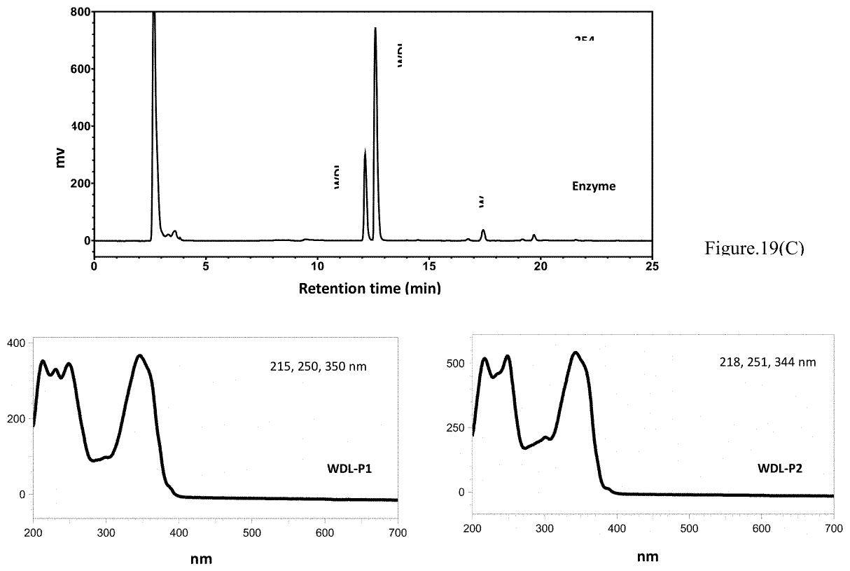

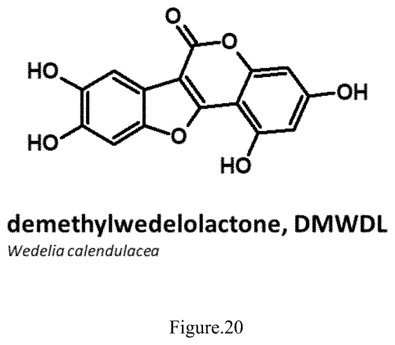

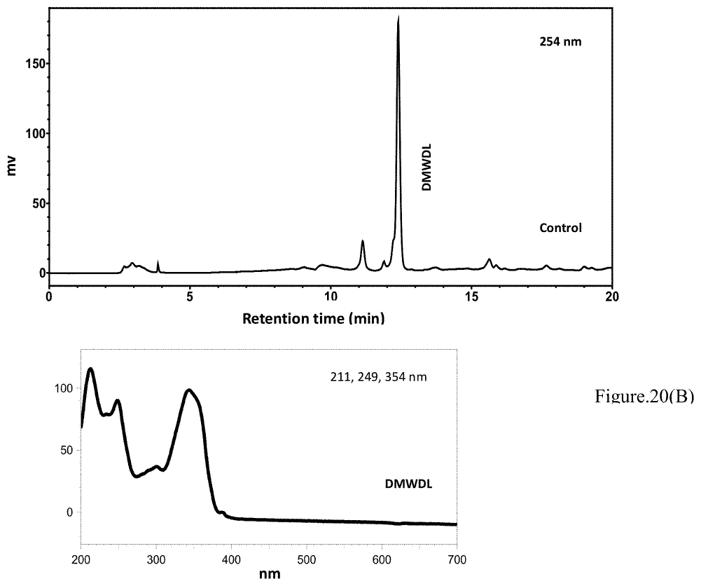

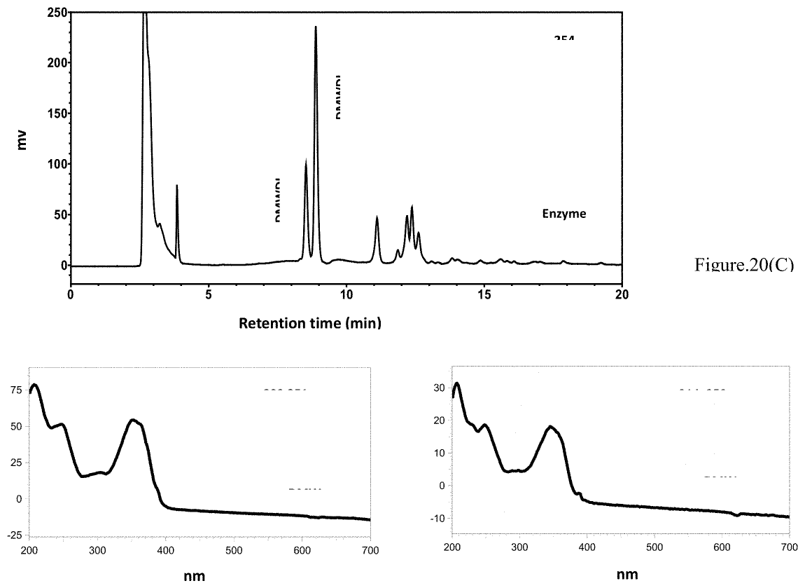

More detailed results are shown in FIG. 2 to FIG. 22.

With regard to curcuminoids, the phosphorylation results of curcumin (CUR), bisdemethoxycurcumin (BDMC), tetrahydrocurcumin (THC), and octahydrocurcumin (OHC) by the recombinant PPSs are shown in FIG. 2 to FIG. 5 respectively.

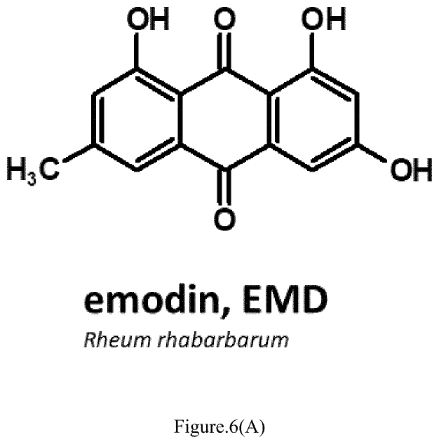

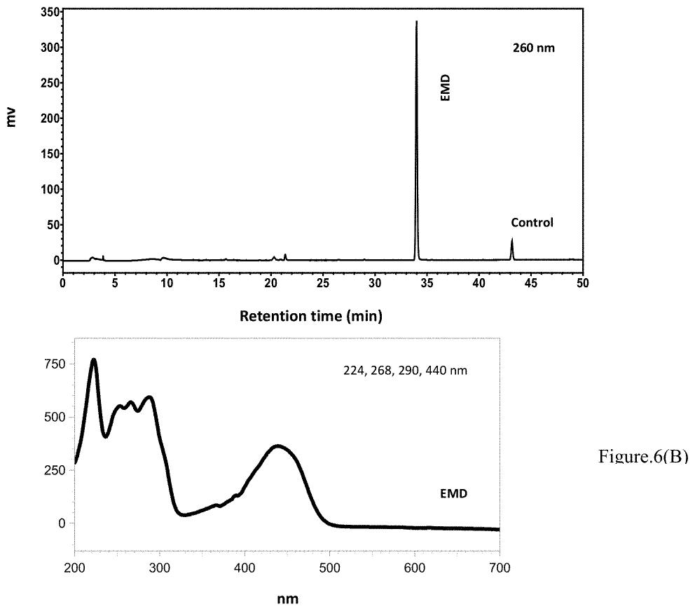

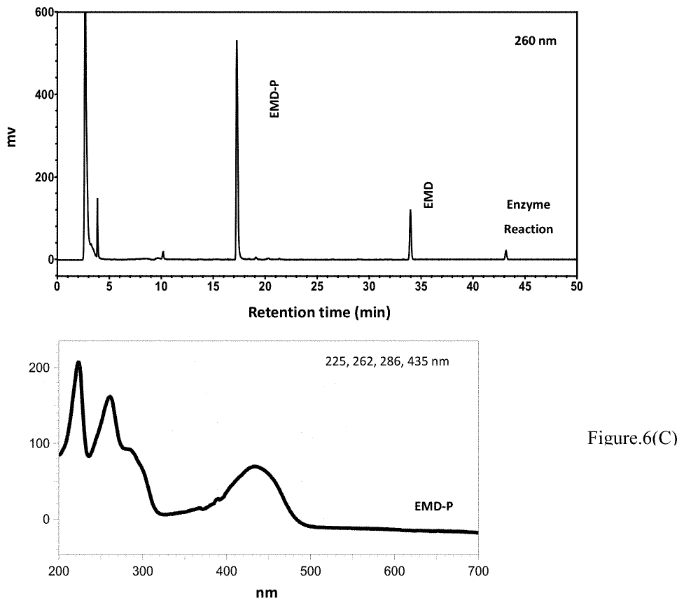

With regard to anthraquinones, the phosphorylation results of emodin (EMD), obtusifolin (OBF), and aloe-emodin (ALE) by the recombinant PPSs are shown in FIG. 6 to FIG. 8 respectively.

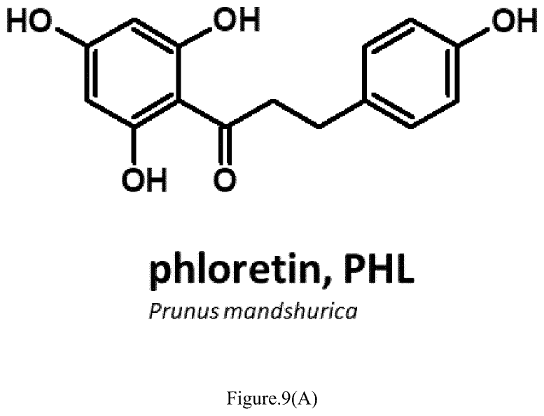

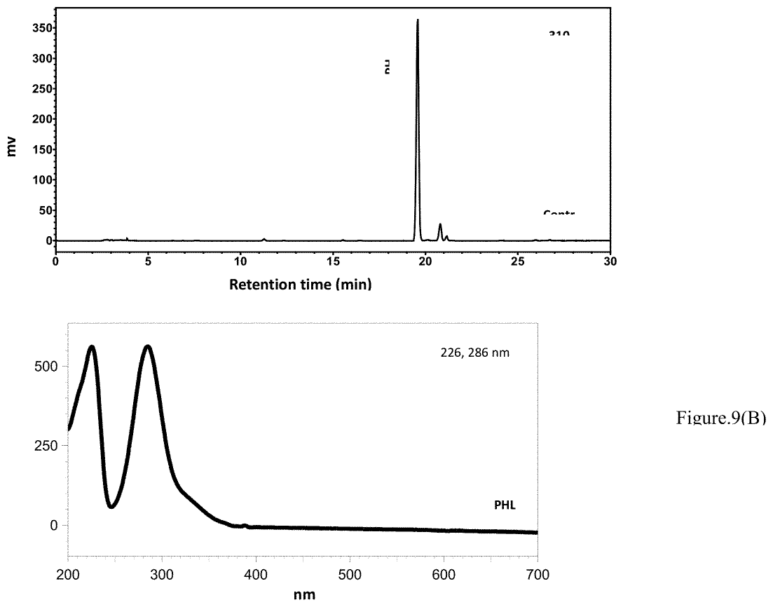

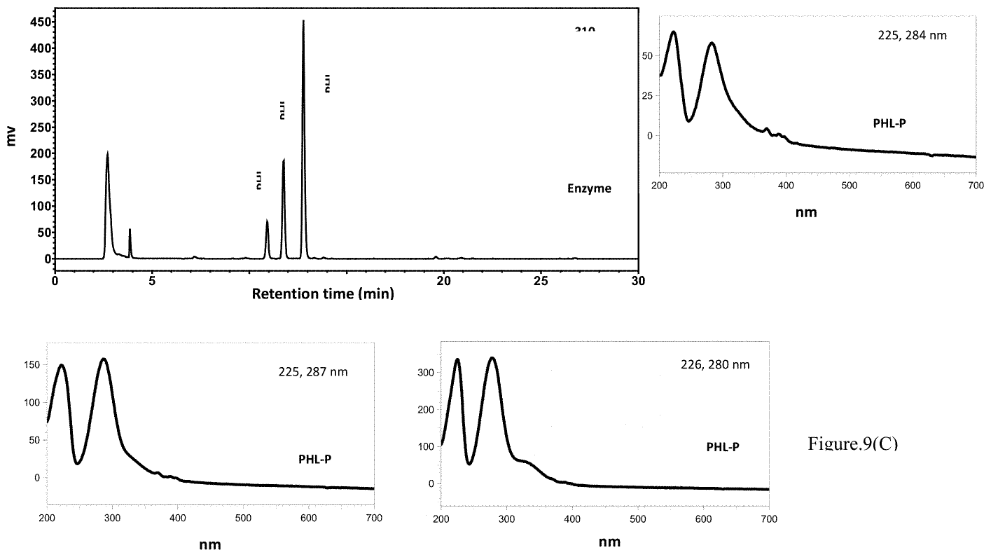

With regard to chalcones, the phosphorylation results of phloretin (PHL), isoliquirtigenin (ISL), and flavokawain A (FKA) by the recombinant PPSs are shown in FIG. 9 to FIG. 11 respectively.

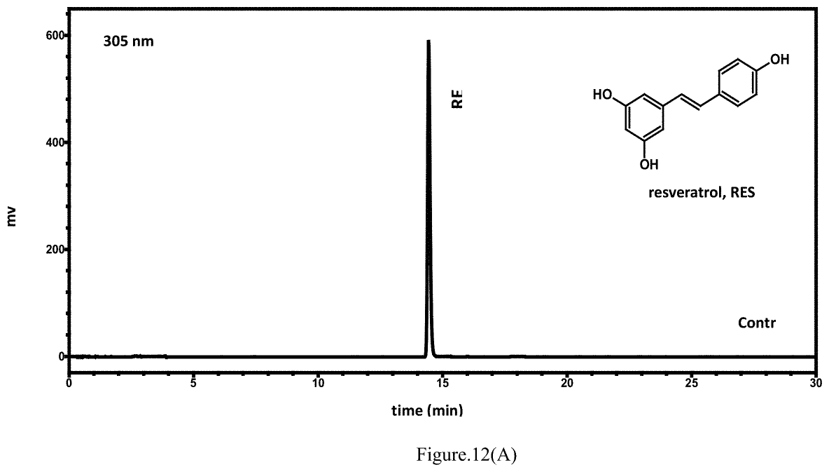

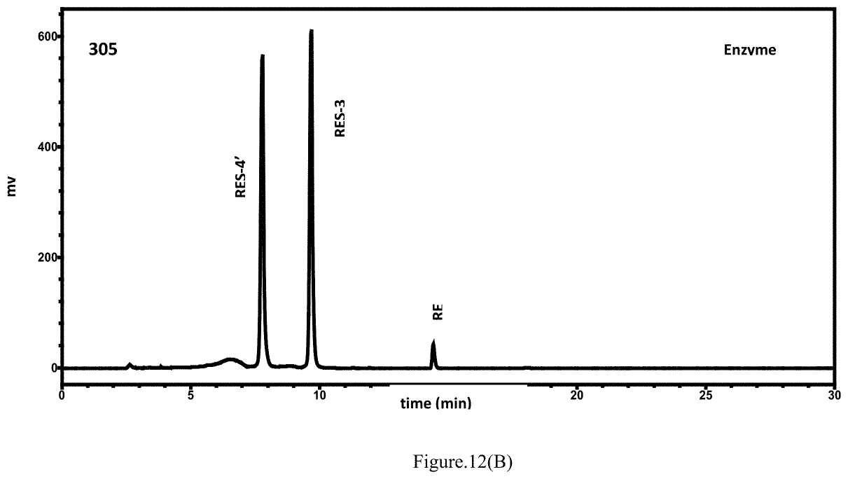

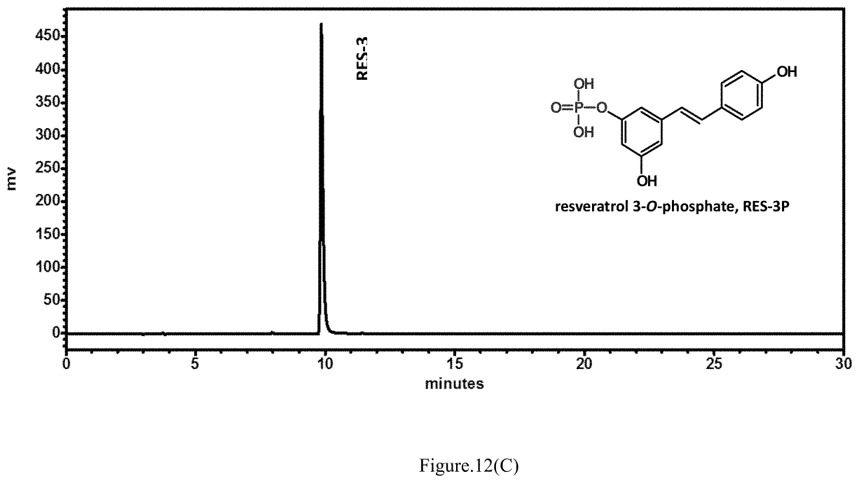

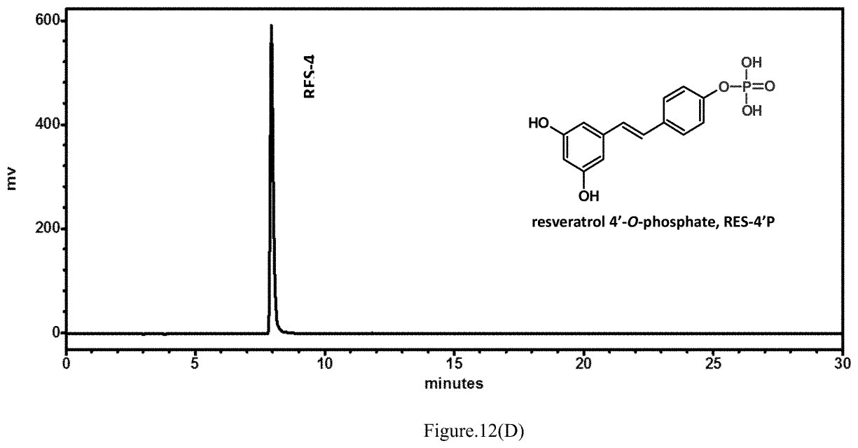

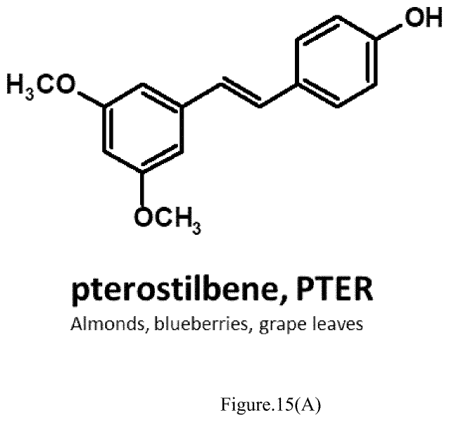

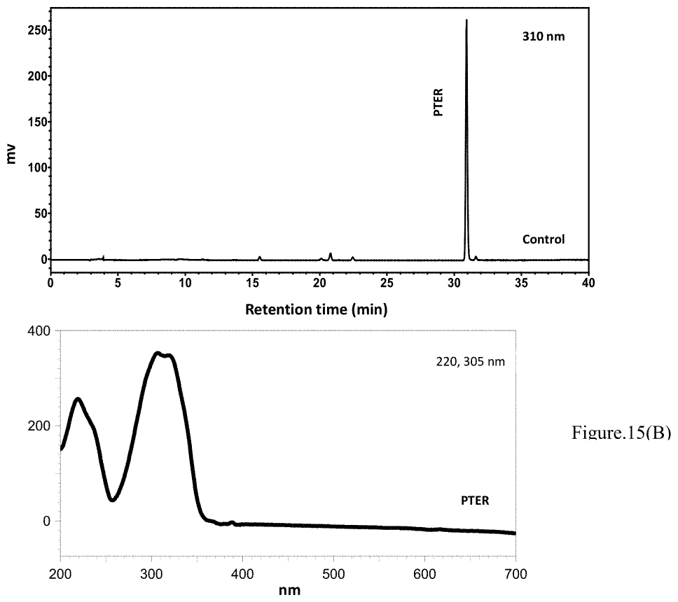

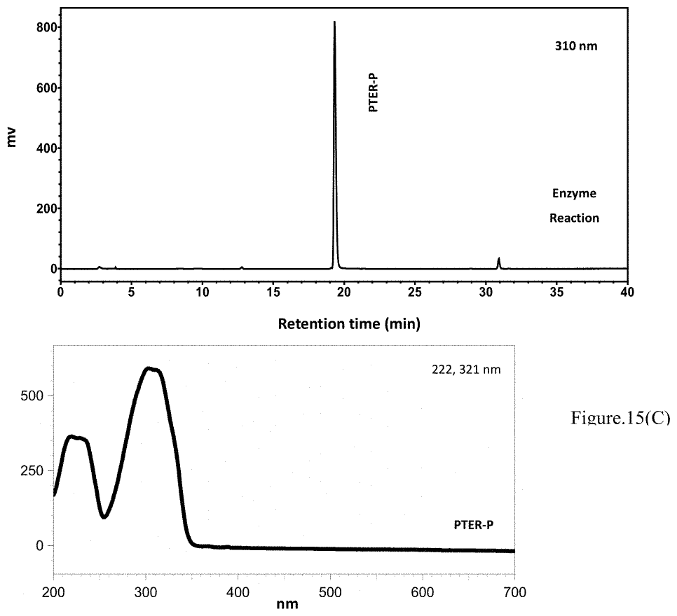

With regard to stilbenes, the phosphorylation results of resveratrol (RES), resveratrol 3-O-phosphate (RES-3P), and resveratrol 4′-O-phosphate (RES-4′P) by the recombinant PPSs are shown in FIG. 12 to FIG. 14 respectively, and the phosphorylation results of pterostilbene (PTER), piceatannol (PCT), and oxyresveratrol (OXY) by the recombinant PPSs are shown in FIG. 15 to FIG. 17 respectively.

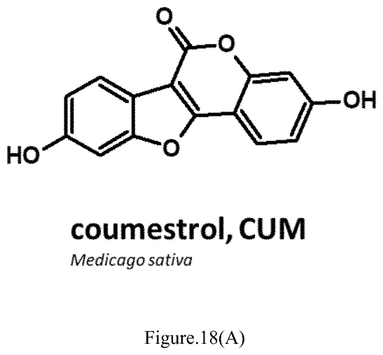

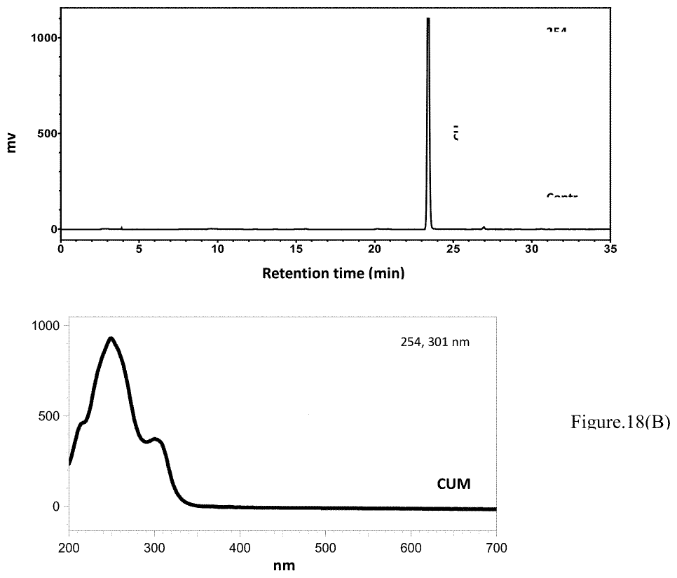

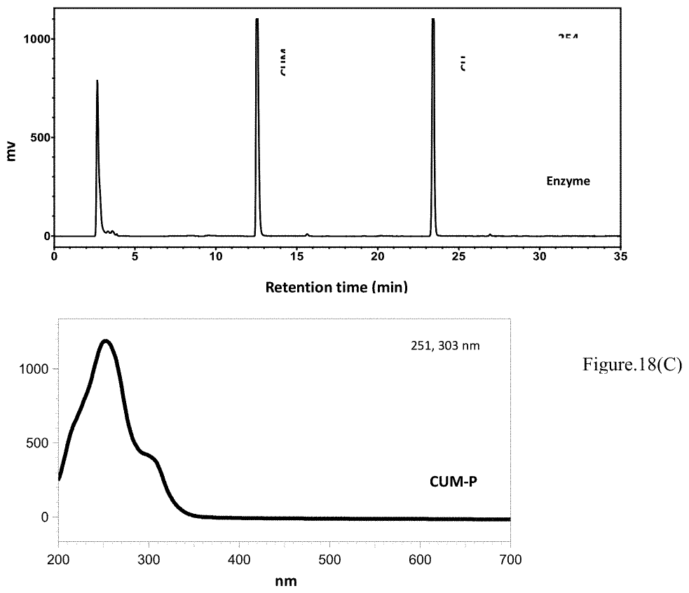

With regard to coumestrols, the phosphorylation results of coumestrol (CUM), wedelolactone (WDL), and demethylwedelolactone (DMWDL) by the recombinant PPSs are shown in FIG. 18 to FIG. 20 respectively.

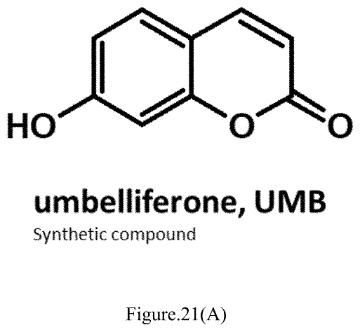

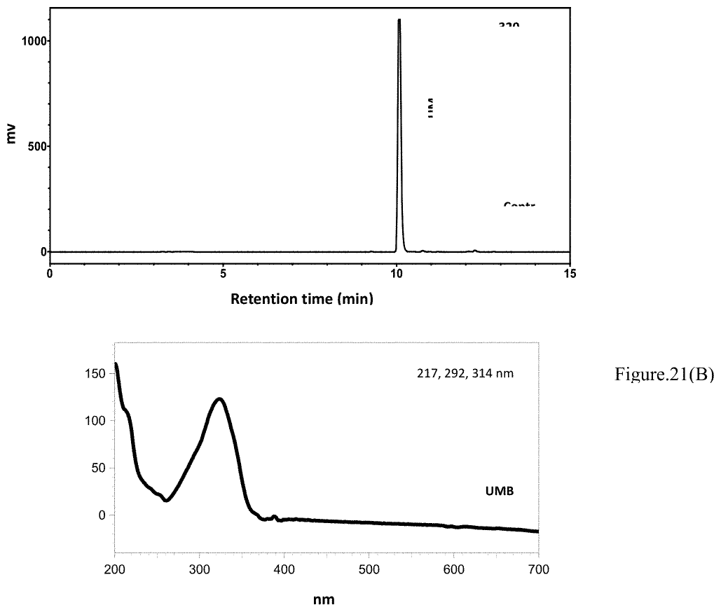

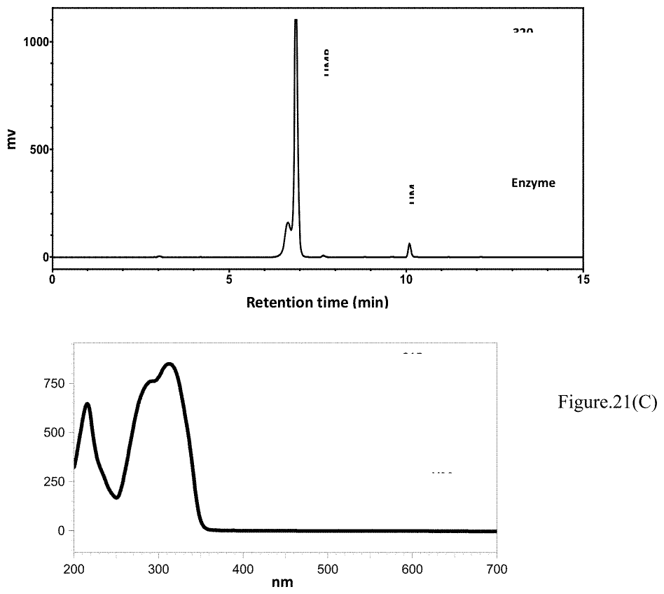

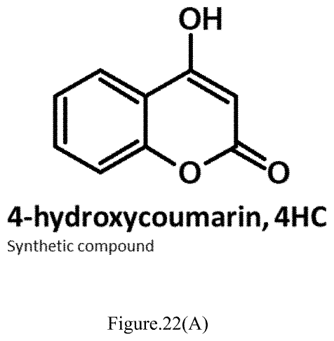

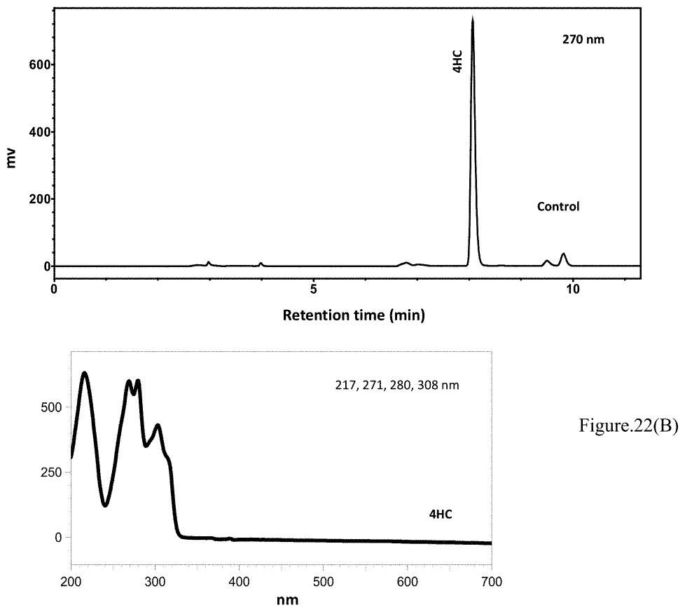

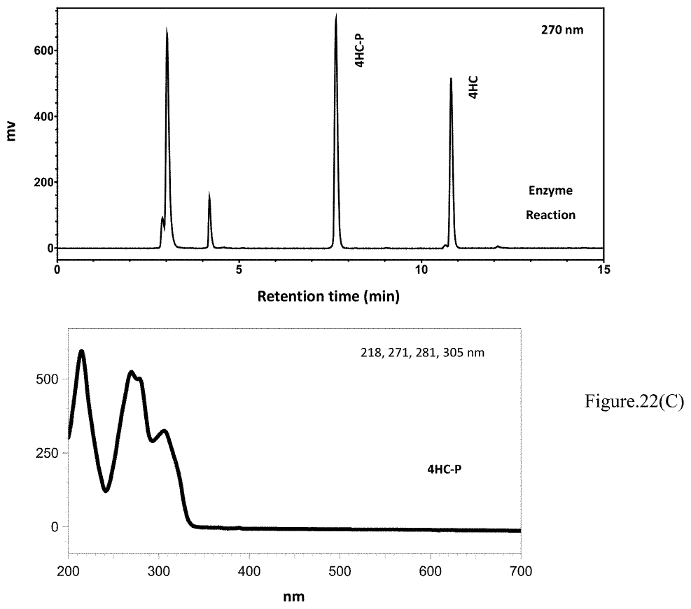

With regard to coumarins, the phosphorylation results of umbelliferone (UMB) and 4-hydroxycoumarin (4HC) by the recombinant PPSs are shown in FIG. 21 and FIG. 22 respectively.

The experimental results show that the recombinant PPSs exhibited an unexpectedly wide promiscuity toward a diversity of species of polyphenolic phytochemicals. The PPSs succeeded in transforming different types of polyphenolic phytochemicals (namely flavonoids, curcuminoids, anthraquinones, chalcones, stilbenes, coumestrols, and coumarins) into their respective phosphate derivatives.

II. Bioinformatic Analysis of the PPS Protein Family

Polyphenolic phytochemical phosphate synthetase (PPS) is referred to in the NCBI database as hypothetical protein BSNT_10146 [Bacillus subtilis subsp. nano BEST195] (BAI87146), its coding gene being yvkC hypothetical protein [Bacillus subtilis subsp. nano BEST195] (gene ID: 14103593), which is composed of 2520 bases. PPS is a novel dikinase (EC 2.7.9.×), whose amino acid sequence has been found, by an analysis based on the InterPro database, to have two conserved domains, namely Glu19-Glu310, which is an ATP-binding domain (IPR002192), and Ile734-Thr834, which is a phosphorylated histidine catalytic domain (IPR008279). From a comparison with the functional domains of pyruvate, phosphate dikinase (PPDK) (EC 2.7.9.1) we further inferred that Lys311-His733 may be a substrate-binding domain, although no classification or research regarding this domain can so far be found in the InterPro database. In this embodiment, a search based on the primary sequence of PPS was conducted in the NCBI database in order to find a homologous protein family, and a bioinformatic analysis was performed to analyze the differences between the primary sequences of the PPS protein family.

1. Comparison of the Sequences of the PPS Protein Family

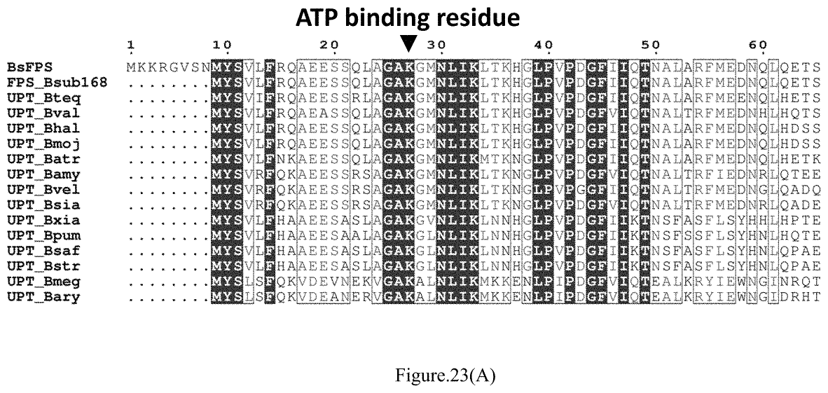

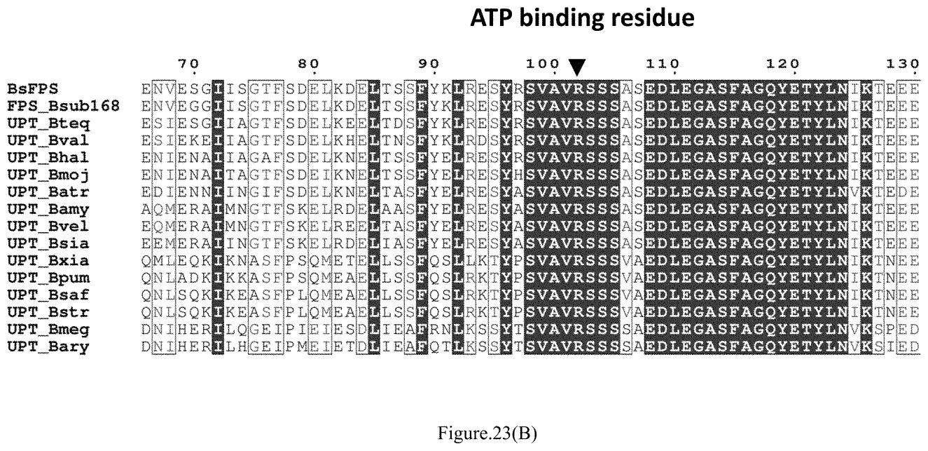

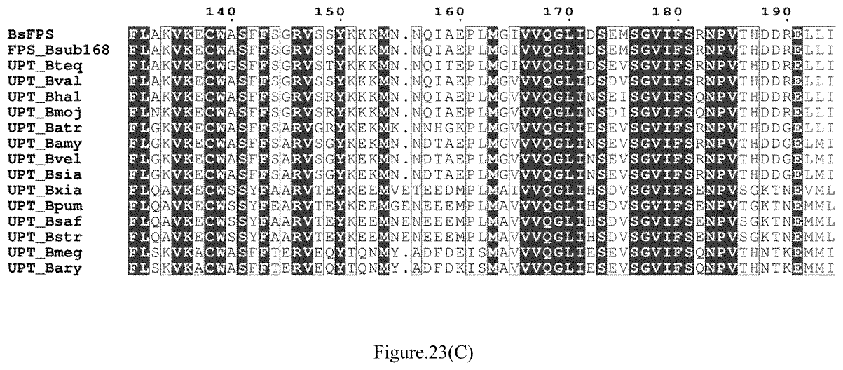

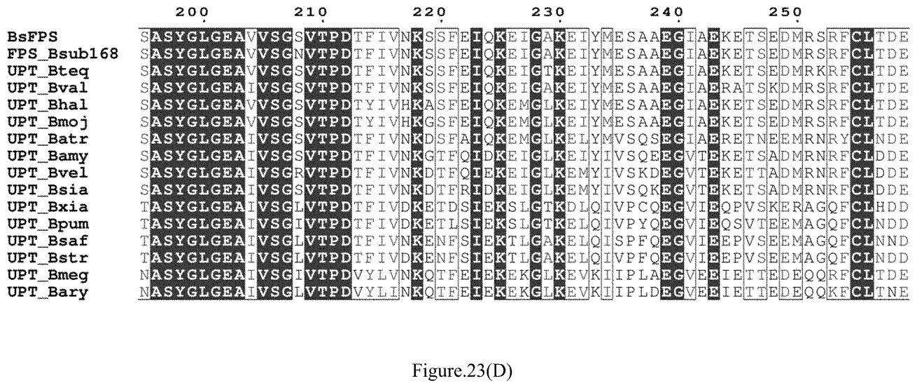

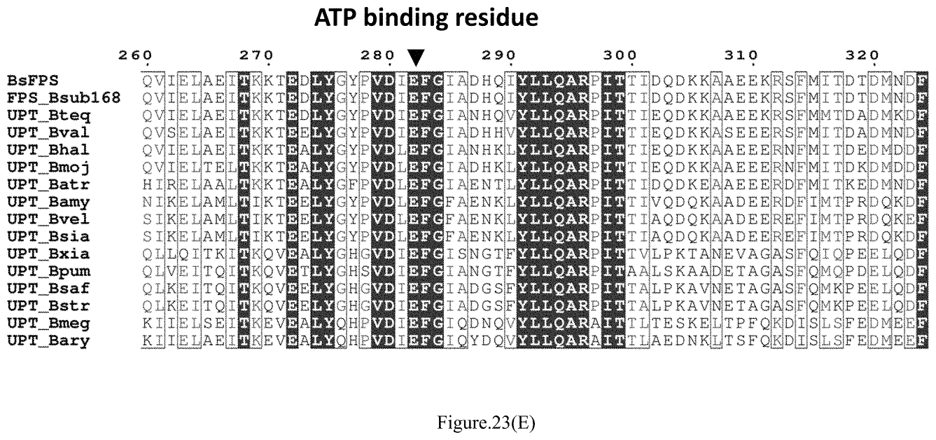

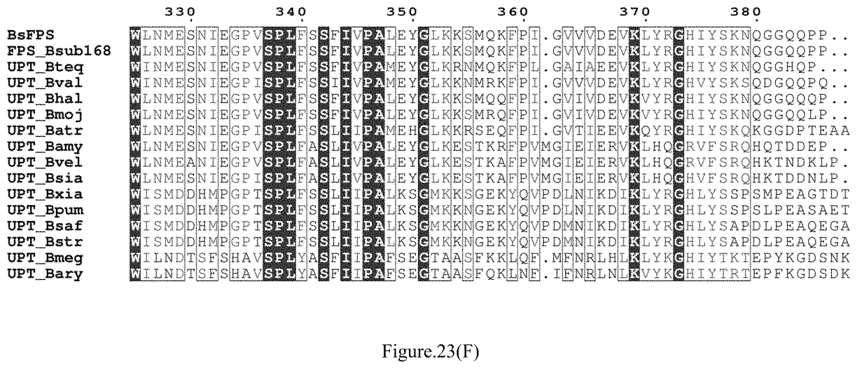

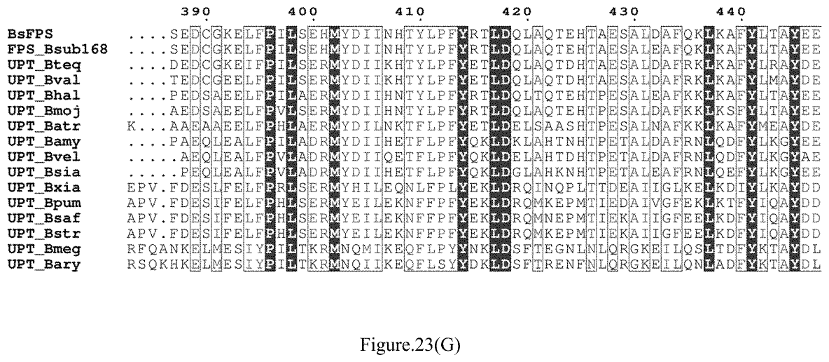

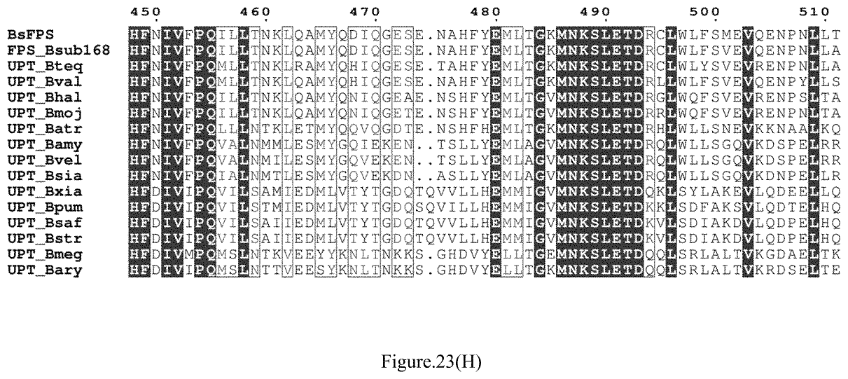

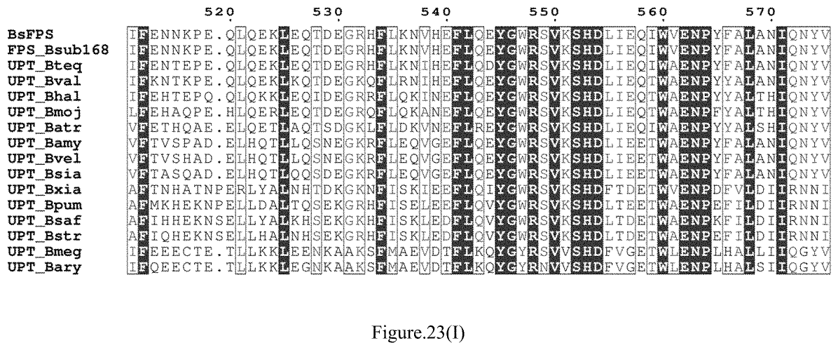

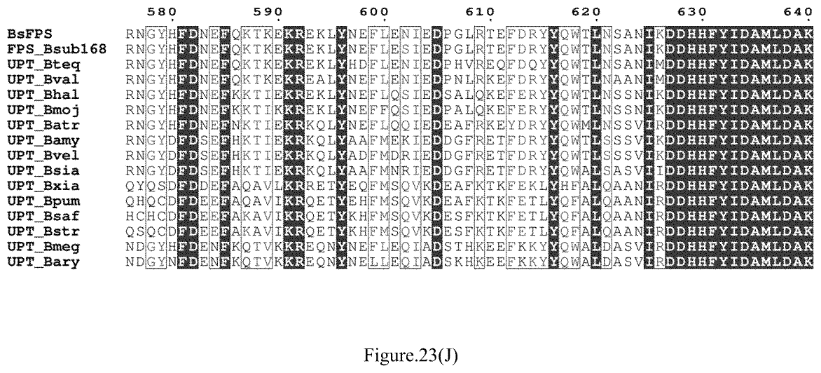

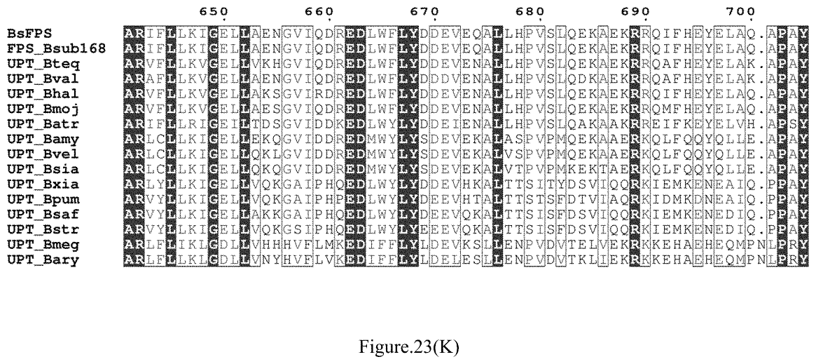

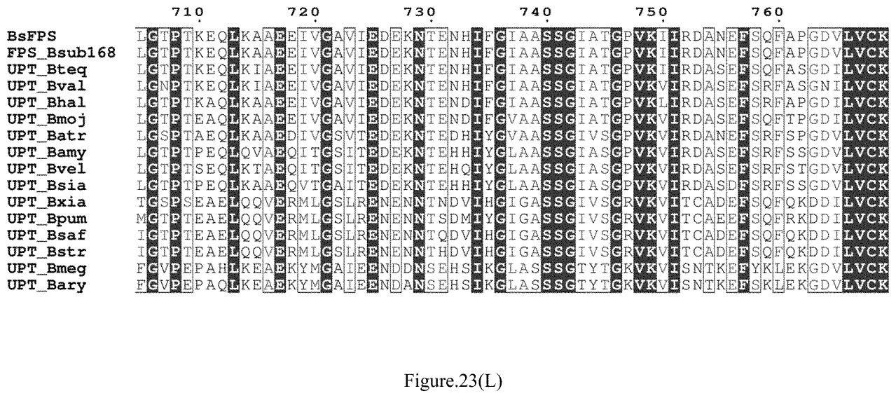

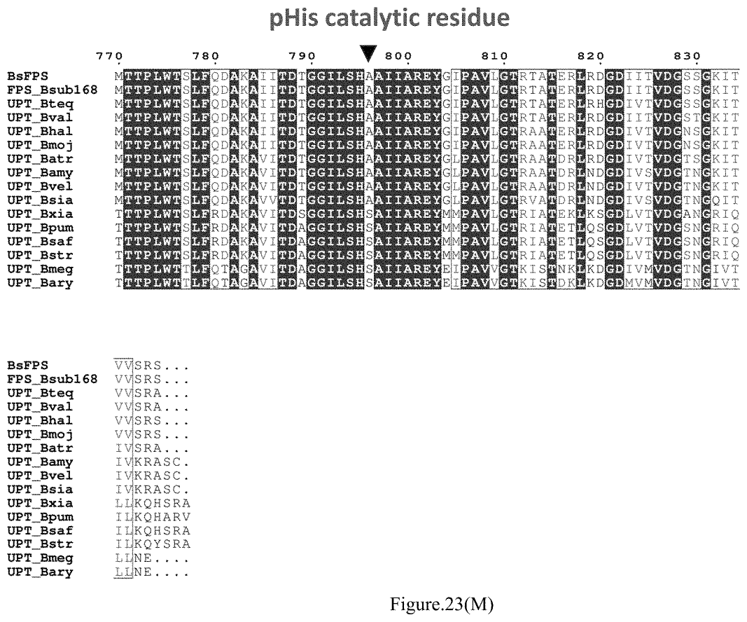

A comparison with the amino acid sequences of the PPSs obtained in this embodiment was conducted with the BLASTp program of the NCBI database to find the most similar 500 sequences, from which the similar amino acid sequences of Bacillus subtilis, Bacillus halotolerans, Bacillus atrophaeus, Bacillus velezensis, Bacillus amyloliquefaciens, Bacillus pumilus, Bacillus safensis, Bacillus aryabhattai, Bacillus megaterium, Aneurinibacillus migulanus, and Paludifilum halophilum were selected, based on a descending order of the degree of identity, for a multiple sequence comparison with the PPSs obtained in this embodiment. The multiple sequence comparison was carried out with ESPript v. 3.0, and the comparison results are shown in FIG. 23. According to the comparison results, both the ATP-binding domain from Glu19 to Glu310 at the N terminal and the phosphorylation catalytic domain from Ile734 to Thr834 at the C terminal are highly conserved, and His795 is an active site for protein phosphorylation and is responsible for catalyzing transfer of the phosphate group such that the phosphorylation catalytic domain can be defined as a phosphorylated histidine catalytic domain.

Interestingly, the substrate-binding domain in the middle shows marked sequence variations, in particular in the amino acid segments Asp302-Gln322 and Lys378-Glu388. It can therefore be inferred from the sequence comparison results that the major differences between homologous protein sequences of different species result from the different sequences of their substrate-binding domains. A highly conserved amino acid segment, however, was found in Ile625-Leu652 of the substrate-binding domain, in particular DDHHFYIDAMLDAKAR (SEQ ID NO: 14). The high conservedness of this sequence indicates that the sequence may be an active site for enzymatic catalysis or an amino acid sequence critical to maintaining the three-dimensional structure.

2. Phylogenetic Tree of the PPS Protein Family

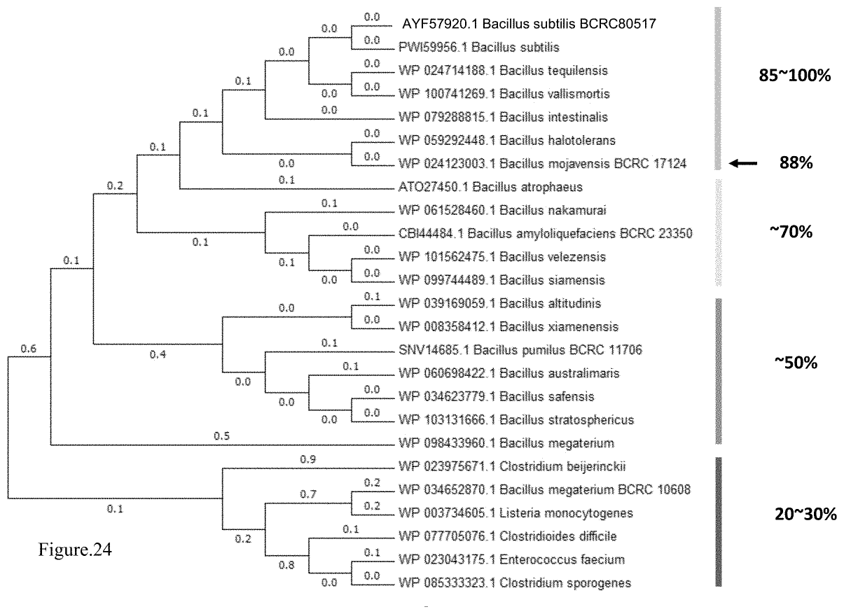

Furthermore, a total of 25 sequences—including those of the PPSs obtained in this embodiment and a number of sequences of different degrees of identity selected from the 500 sequences found with the BLASTp program of the NCBI database through a comparison with the amino acid sequences of the PPSs obtained in this embodiment, plus the sequence of LmRPH (EC 2.7.9.6)—were compared against one another to create the phylogenetic tree shown in FIG. 24. The tree was constructed with MEGA 6. More specifically, ClustalW was used to compare the whole amino acid sequences, and then the neighbor-joining method was used to establish the evolution relationship. As shown in FIG. 24, the more similar to the sequence of the PPS of interest in the present invention a homologous protein sequence is, the closer the phylogenetic relationship between them. It is worth particular notice that the foregoing recombinant PPS genes were cloned from B. subtilis BCRC 80517 (the expressed protein being BsPPS, which is the PPS of interest in the invention), B. amyloliquefaciens BCRC 23350 (the expressed protein being BmojPPS), and B. mojavensis BCRC 17124 (the expressed protein being BamyPPS) respectively, and that the degrees of identity of the recombinant PPS genes cloned from the latter two bacteria to the recombinant gene cloned from the first bacterium are not lower than 70% (see FIG. 24 and Table 1). It can therefore be inferred that a polypeptide having an amino acid sequence whose degree of identity to the amino acid sequence of the PPS of interest in the invention is not lower than 70% has the enzymatic activity for catalyzing binding of the foregoing substrates.

TABLE 1

| | | | | | | | Phosphorylated | |
|---|---|---|---|---|---|---|---|---|
| | | | ATP-binding | | Substrate-binding | | histidine catalytic | |
| | Whole sequence | | domain | | domain | | domain | |
| | Degree of | Degree of | Degree of | Degree of | Degree of | Degree of | Degree of | Degree of |
| Homologous | identity | similarity | identity | similarity | identity | similarity | identity | similarity |
| protein | (%) | (%) | (%) | (%) | %) | (%) | (%) | (%) |
| BsPPS | 100 | 100 | 100 | 100 | 100 | 100 | 100 | 100 |
| BmojPPS | 87.4 | 90.6 | 89.4 | 93.4 | 84.8 | 88.2 | 93.8 | 96.3 |
| BamyPPS | 70.1 | 76.7 | 77.5 | 82.5 | 63.8 | 71.0 | 82.5 | 91.3 |

III. Simulation of the PPS Protein Structure

The experiment described in this section aimed to predict a possible tertiary structure of the PPS of interest in the present invention by homology modeling, or more specifically by SWISS-MODEL, and to predict possible important catalytic sites of the PPS of interest according to a comparison with the template structures.

1. Search for a Template with which to Establish the Simulated Structure

An online analysis of the protein sequence of the PPS of interest in the present invention was carried out through SWISS-MODEL in order to search the SWISS-MODEL Repository for a known protein structure with a similar sequence that could serve as a template. Currently, the most similar known structure to the PPS of interest is rifampin phosphotransferase from Listeria monocytogenes (LmRPH), i.e., EC 2.7.9.6 (Qi et al., 2016, Peter J. Stogios et al., 2016), which has a degree of identity of about 26%, a coverage of 95%, and a total of six structures, each co-crystallized with a different ligand, as shown in its structure data in the PDB database. In the six structures, 5FBU, 5FBT, 5FBS, and 5HV1 are structures with a stable phosphorylated histidine catalytic domain and a stable substrate-binding domain, and 5HV2 and 5HV3 are structures with a stable ATP-binding domain and a stable substrate-binding domain. Each of the six structures was used as a template with which to predict the tertiary structure of the PPS of interest by homology modeling.

2. Establishment and Analysis of the Simulated PPS Structure

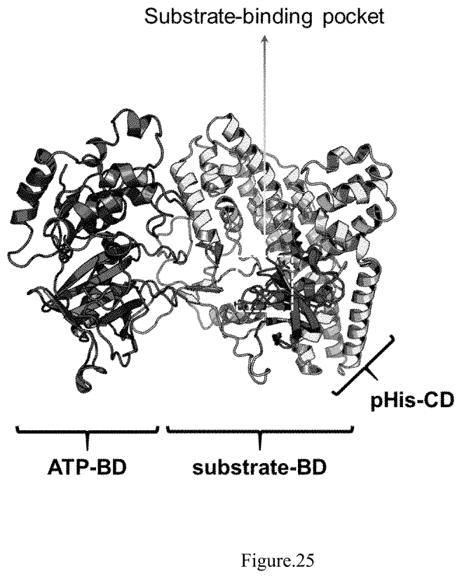

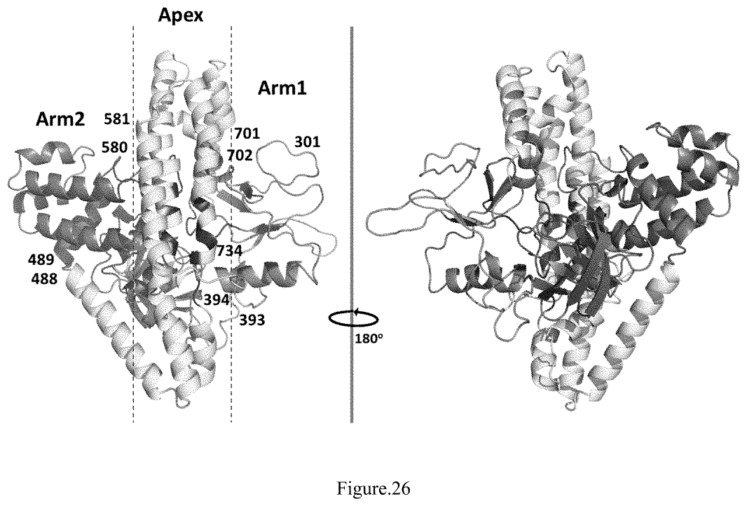

Once the tertiary structures were constructed with the templates by homology modeling respectively, their Qualitative Model Energy ANalysis (QMEAN) values were calculated with software. The QMEAN value is a composite score for the distribution of the major geometrical structures of a simulated protein structure and has a range of reliability from 0 to −4. The closer to 0, the more reliable the simulation result. As we intended for the phosphorylated histidine catalytic domain to swing to the stable structure of the substrate-binding domain, thereby allowing the catalytically active domain around His795, which is known to be responsible for catalyzing transfer of the phosphate group, to be found, the template-based simulated structure with the lowest QMEAN value, i.e., the simulated PPS structure based on the template 5FBT (QMEAN=−4.06), was selected for use, and the selected structure was observed with PyMOL. The simulated PPS structure was then divided as follows with reference to the functional domains of the structure of the LmRPH template: 1Met-Thr300 was defined as the ATP-binding domain (ABD), 301Thr-His733 as the substrate-binding domain (SBD), and Ile734-Ser839 as the phosphorylated histidine catalytic domain (pHis-CD), as shown in FIG. 25. ABD and pHis-CD are highly conserved in the primary sequence, and their structures and catalysis mechanisms are known. To better study the structure of SBD, SBD was particularly extracted to facilitate observation of its structural details. After an observation and comparison of the secondary structures of LmRPH and of the simulated PPS structure, SBD was further divided into Arm1 (Thr301-Lys392, Ala701-His733), Apex (Glu393-Asn487, His580-Gln700), and Arm2 (Lys488-Tyr579) as shown in FIG. 26 and was viewed as having a cross shape, with Apex serving as the central axis, and Arm1 and Arm2 located on two lateral sides of Apex respectively and enclosing HD in an “embracing” manner such that a hydrophobic pocket is formed therebetween. The hydrophobic pocket might be a catalytically active site of the PPS.

IV. Analysis of Important Catalytically Active Sites of the PPS of Interest

1. Prediction of Important Catalytically Active Sites of the PPS of Interest

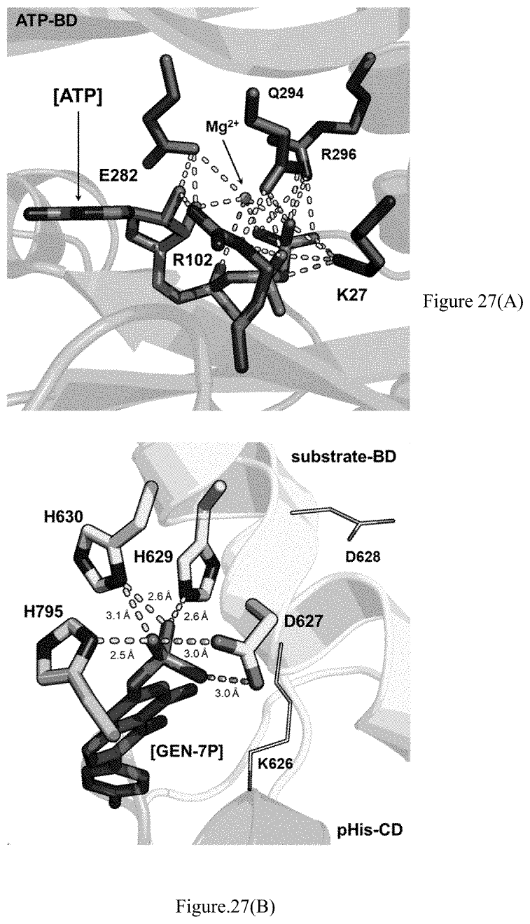

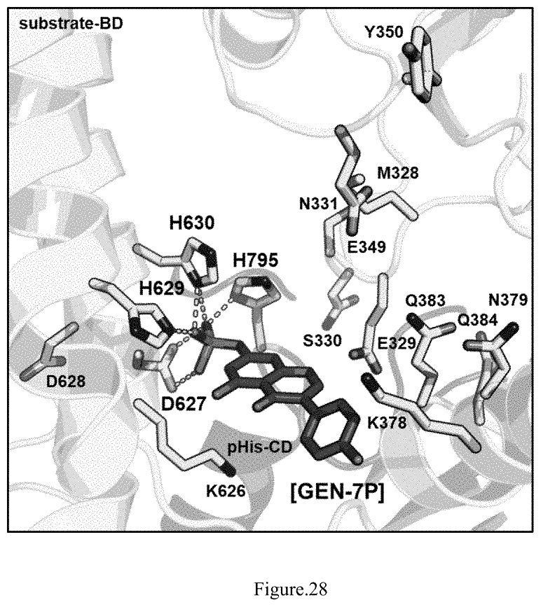

To begin with, we compared the primary sequences of the ATP-binding domains and phosphorylated histidine catalytic domains of the PPS of interest in the present invention and of LmRPH and found two important catalytic sites, namely Lys27, which is related to ATP binding, and His795, which is responsible for carrying and transferring the phosphate group. To find an important catalytic site in the substrate-binding domain, the amino acids around the His795 site (which is responsible for carrying the phosphate group to facilitate catalysis) of the simulated PPS structure were observed in greater detail and were compared with the major amino acids around His825 (which is known to have important functions) of the LmRPH template. The active rifampin-phosphorylation-catalyzing domain of LmRPH is such that after His825 carries the phosphate group from ATP to the substrate-binding domain, the transferred phosphate group is stabilized by the electrically charged or polar amino acids of Arg666, Glu667, Lys670, and Gln337. Moreover, the Apex of LmRPH has a highly conserved REXXK motif, which is an important catalytically active site. Interestingly, the Apex structure of the simulated PPS structure also has a highly conserved segment, or more specifically a continuous electrically charged KDDHH segment consisting of Lys626, Asp627, Asp628, His629, and His630. A structural observation reveals that this segment is also located around the phosphorylation site His795 and forms a tightly enclosed structure as shown in FIG. 27. It was also found that Met328, Glu329, Ser330, and Asn331 in Arm1 of the simulated PPS not only might correspond in position to Gln337 of LmRPH, but also are similar to Gln337 in being polar. In addition, we searched outward from this conjectural active domain in order to find more active sites that may be related to substrate binding, and a structural observation plus the comparison results in section II regarding the activity of different homologous protein sequences toward different substrates led to the following findings: Glu349, Tyr350, Lys378, Asn379, Gln383, and Gln384 in Arm1, which amino acids have significantly different sequences from one another as shown in FIG. 28, might be the amino acids in the PPS of interest that are related to substrate binding.

2. Verification of the Predicted Important Catalytic Sites by Performing Site-Directed Mutagenesis on the PPS of Interest

2-1 Creation of Site-Directed Mutant Strains of the PPS of Interest

To verify whether the amino acids predicted from the simulated PPS structure are important catalytic sites, site-directed mutagenesis was conducted by substituting an amino acid under discussion with an amino acid with a different property (e.g., by substituting the electrically charged Lys or the polar Asn with Val, which is neither electrically charged nor polar) and thereby changing the catalytic activity of the protein. Generally, an amino acid of interest is substituted with alanine (Ala), which is the smallest residue, in order to change the charge state or polarity of the site of the amino acid and thus deprive the protein of its ability to bind with a substrate. We performed site-directed mutagenesis on the predicted catalytic sites stated in the previous paragraph by substituting those amino acids with Ala, and the following site-directed mutant strains of the PPS of interest in the present invention were created as a result: H795A, K27A, K626A, D627A, D628A, H629A, H630A, M328A, E329A, S330A, N331A, E282A, Y350A, K378A, N379A, R102A, R296A, Q294A, Q383A, and Q384A.

More specifically, the previously constructed PPS expression plasmid pps_Bsub::pET47b(+) was subjected to a polymerase chain reaction (PCR) into which a mismatched primer set was introduced. After that, Dpnl was used to catalyze decomposition of the plasmid template, which was methylated in the first place. The mutant plasmid mutant-pps_Bsub:: pET47b(+) was then cloned in E. coli DH5α by way of transformation, and the cloned mutant-pps_Bsub:: pET47b(+) was extracted with a plasmid DNA extraction kit and sent to Genomics Bioscience & Technology Co., Ltd. for sequencing. The mutant-pps_Bsub:: pET47b(+) whose sequence had been ascertained was then expressed in E. coli BL21 (DE3) by way of transformation.

2-2 Expression of the PPS of Interest and its Protein Mutant Strains

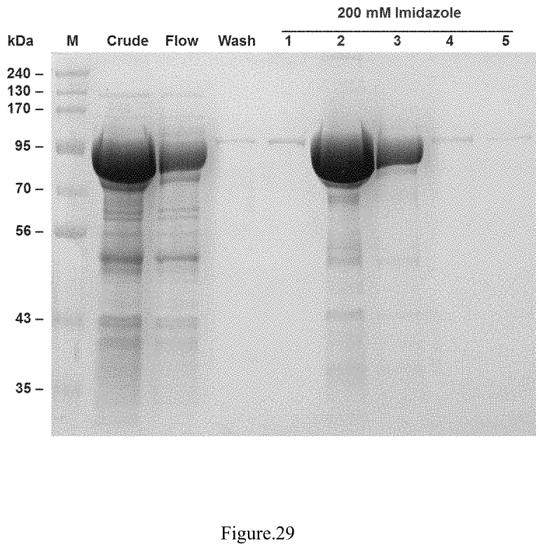

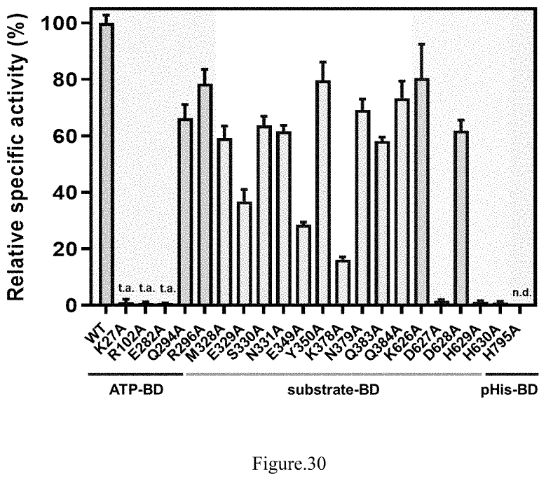

The protein expression strain pps_Bsub::pET47b (+)/BL21 (DE3) was cultured and added with IPTG to induce its expression. The resulting bacterium body was subjected to ultrasonic disruption to produce a crude enzyme extract. The crude enzyme extract underwent preliminary purification in a HisTrap™ HP column and was collected in a fraction collector, before the PPS expression was analyzed by SDS-PAGE. The analysis results are shown in FIG. 29. The crude enzyme extract had a total protein concentration of 10 mg/mL and was overexpressed beyond the bearing capacity of the column such that while the sample was being injected, a large amount of protein flowed out without being adsorbed. When the unadsorbed protein was washed with a buffer solution containing 40 mM imidazole, only a light-color band showed. When eluting in the end with a buffer solution containing 200 mM imidazole, however, a thick noticeable band was observed at 95 kDa for the second and the third columns, indicating the presence of the PPS. The eluents of the two columns were combined and subjected to desalting chromatography in a HiTrap desalting column, with the protein concentration eventually diluted to 1 mg/mL as analyzed by the Bradford method. The relative specific activity of the PPS protein mutant strains is plotted in FIG. 30.

2-3 Determination of the Enzymatic Activity of the PPS of Interest and its Protein Mutant Strains (with Genistein Used as the Catalyst Substrate in this Embodiment)

Once an enzyme was prepared, its protein concentration was diluted to 0.1 mg/mL. The enzyme and the enzyme reaction liquid were mixed at a ratio of 1:1 (v/v) and were allowed to react at 40° C. for 10 minutes, at the end of which methanol was added to dilute the protein tenfold and thereby terminate the reaction. The changes in concentration of genistein (GEN) and G7P (a product of GEN phosphorylation) were analyzed. The catalytic activity of the PPS of interest in the present invention and its mutant strains were subsequently calculated and compared, as shown in FIG. 30. According to the calculation and comparison results, the K27A protein mutant strain lost its activity because its ability to bind with ATP was destroyed, H795A exhibited a total loss of PPS activity because of its incapability to carry and transfer the phosphate group, Lys27 and His795 were two important catalytic sites in the ATP-binding domain, and Arg102 and G282 were also important sites in the ATP-binding domain. As to the predicted catalytic sites in the substrate-binding domain, D627A, H629A, and H630A exhibited a significant decrease in protein activity; had little, if any, activity for catalyzing binding of genistein; and were important catalytic sites (i.e., D627, H629, and H630) in the active domain for stabilizing the phosphate group. The activity test results of other protein mutant strains show no total loss of activity but a trend of decrease in activity. In particular, E329A and K378A exhibited a relatively significant decrease in activity and might be amino acids that bound to and thereby stabilized genistein. However, while the aforesaid important catalytic sites in the active domain for stabilizing the phosphate group were inferred with relatively great certainty, the amino acids in Arm1 either had relatively poor prediction results or had important catalytic sites composed of non-polar amino acids (which when substituted with Ala produce little change in property) such that the combined use of site-directed mutagenesis and enzymatic activity determination lacked accuracy.

2-4 Circular Dichroism Spectra of the PPS of Interest and its Protein Mutant Strains

Circular dichroism spectroscopy is a method commonly used to investigate protein structures. As the α-helix, β-sheet, and random-coil conformations have different waveforms in a far-UV (with wavelengths ranging from 200 to 250 nm) circular dichroism spectrum, whether the secondary structure of a protein has changed can be determined according to whether the waveforms are changed.

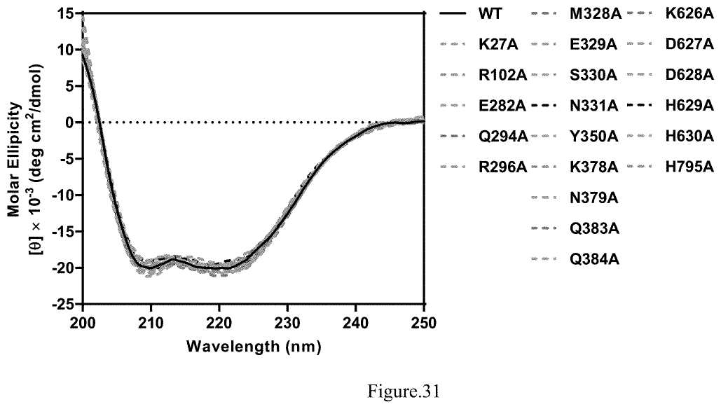

While the importance of the aforesaid important catalytic sites to catalysis has been determined by site-directed mutagenesis and enzymatic activity determination, the change in enzymatic activity of a protein mutant strain is not necessarily the result of a change in its three-dimensional structure caused by amino acid substitution. A circular dichroism spectrum helps us observe the three-dimensional structure of a protein so that by measuring the far-UV circular dichroism spectra of the PPS of interest in the present invention and its protein mutant strains, it can be seen whether it was a change in the secondary structure of a PPS mutant strain that effected a change in its activity. Referring to FIG. 31, the PPS protein mutant strains have substantially identical waveforms in the far-UV spectra, and those waveforms resemble that of the α-helix conformation, meaning the major secondary structure of the PPS protein mutant strains is a β-helix. This observation is consistent with the simulation results.

Conclusion: The homologous genes pps and homologous proteins PPS of the polyphenolic phytochemical phosphate synthetase (PPS) found in Bacillus subtilis BCRC 80517 in this embodiment exist extensively in various procaryotes. Those homologous proteins have catalysis mechanisms partly similar to those of PPDK (EC 2.7.9.1) and LmRPH (EC 2.7.9.6), include highly homologous ATP-binding domains and phosphorylated histidine catalytic domains, but differ greatly in their substrate-binding domain sequences.

It can be inferred from the enzymatic catalysis results of the PPSs derived respectively from B. subtilis BCRC 80517, B. amyloliquefaciens BCRC 23350, and B. mojavensis BCRC 17124 that a polypeptide whose amino acid sequence has a 70% or higher degree of identity to the amino acid sequence of the PPS of interest in the present invention has the enzymatic activity for catalyzing the biotransformation of a substrate.

The tertiary structure of the PPS of interest in the present invention was successfully simulated by homology modeling, or more specifically by SWISS-MODEL. The structures of the ATP-binding domain and of the phosphorylated histidine catalytic domain were predicted with relatively high accuracy because both domains are high conserved. The substrate-binding domain has a cross-shaped structure, with Apex forming the central axis, and Arm1 and Arm2 located on two lateral sides of Apex respectively and enclosing the phosphorylated histidine catalytic domain to form a catalytically active domain. Possible important catalytic sites of the PPS of interest were predicted from a comparison between the simulated structures and their templates.

PPS protein mutant strains were created by site-directed mutagenesis, which together with an analysis of the activity of enzymatic catalysis and circular dichroism spectroscopy (which ascertained the secondary structure) proved that Lys27 and His795 are important catalytic sites in the ATP-binding domain and in the phosphorylated histidine catalytic domain respectively, and that Asp627, His629, and His630 are important catalytic sites for stabilizing the phosphate group-binding domain at His795.

According to the above, the present invention has ascertained important catalytic sites of a polypeptide so that the polypeptide can be provided with a continuous electrically charged amino acid segment at a specific position while being separated or engineered, and that when the polypeptide or a microbe having the nucleic acid sequence coding for the polypeptide is used to synthesize a polyphenolic phytochemical phosphate, the success rate of polyphenolic phytochemical phosphorylation or the yield of the synthetic polyphenolic phytochemical phosphate derivative will be increased in comparison with that of the prior art.

What is claimed is:

1. An isolated or engineered polypeptide comprising a homologous protein sequence that is more than 70% identical to the phenolic phytochemical phosphate synthetase (SEQ ID NO: 13); wherein said polypeptide comprises a conserved domain which is based on the phenolic phytochemical phosphate synthetase (SEQ ID NO: 13) and sequentially comprises:
an ATP-binding domain, comprising active catalytic sites of Lys27, Arg102, and Glu282;
a substrate-binding domain, comprising a conserved motif of DDHHFYIDAMLDAKAR (SEQ ID NO: 14), and comprising active catalytic sites of Asp627, His629, and His630;
and a phosphorylated histidine catalytic domain, comprising His795.

2. The polypeptide according to claim 1, wherein the substrate-binding domain comprises a homologous protein sequence that is more than 70% identical to the amino acid sequence of SEQ ID NO: 1.

3. The polypeptide according to claim 1, wherein the polypeptide catalyzes a substrate to its phosphate derivative, and said substrate is a phenolic phytochemical.

4. The polypeptide according to claim 3, wherein the substrate is selected from the group consisting of the following formulas:
Ar1-L-Ar2 (I), 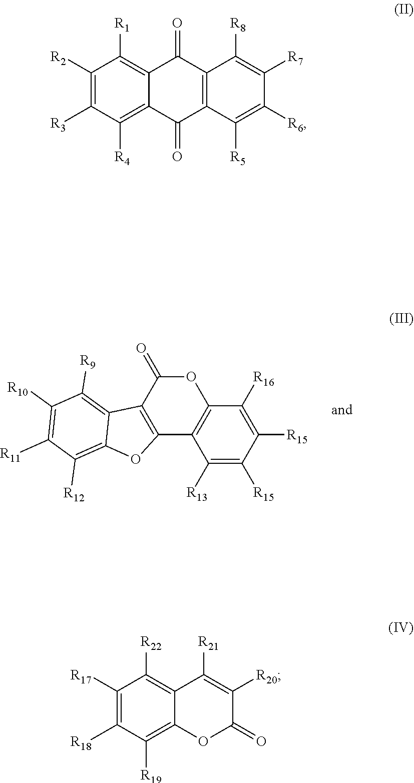
wherein Ar1 is an aryl group of the following formula: 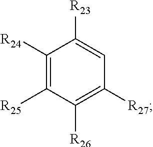
Ar2 is an aryl group of the following formula: 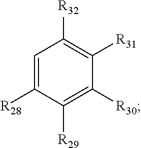
L is a linking group comprising 3 to 7 backbone carbon atoms forming a chain linking Ar1 and Ar2 as the case may be, wherein L comprises at least one of a double bond, a carbonyl group and a hydroxyl group;
R₁ to R₈ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₁ to R₈ is a hydroxyl group;
R₉ to R₁₆ are respectively H, (C₁-C₅)alkyl group, hydroxyl group, OR₃₃, OCH₂OR₃₄, OCOR₃₅, COR₃₆, CO₂R₃₇, OCH₂COOR₃₈, OCH₂(OR₃₉)₂, OC═ONHR₄₀, halogen, nitro, amino, NR₄₁R₄₂, cyano group, mercapto group, SR₄₃, S(O)_{q}R₄₄, (C₁-C₅)chloroalkyl group, (C₁-C₅)haloalkoxy group, (C₂-C₆)alkenyl group, (C₂-C₆)alkynyl group, (C₃-C₁₀)cycloalkyl group, (C₆-C₁₁)phenyl group or (C₇-C₁₂)benzyl group, wherein q is an integral of 1 to 3, and at least one of R₉ to R₁₆ is a hydroxyl group;
R₁₇ to R₂₂ are respectively H, methoxy group or hydroxyl group, and at least one of R₁₇ to R₂₂ is a hydroxyl group, or Rao and R₂₁, R₁₇ and R₁₈, R₁₇ and R₂₂, R₁₈ and R₁₉ or their combination are fused to form a (C₃-C₆)cycloalkyl group with hydroxyl group or a (C₆-C₁₀)aryl group with hydroxyl group;
R₂₃ to R₂₇ are respectively H, methoxy group or hydroxyl group, and at least one of R₂₃ to R₂₇ is a hydroxyl group;
R₂₈ to R₃₂ are respectively H, methoxy group or hydroxyl group, and at least one of R₂₈ to R₃₂ is a hydroxyl group;
R₃₃ to R₃₄ are respectively (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₅ is (C₁-C₅)alkyl group, (C₁-C₅)haloalkoxy group, (C₆-C₁₁)phenyl group or (C₇-C₁₂)benzyl group;
R₃₆ is (C₁-C₅)alkyl group, (C₁-C₅)haloalkoxy group, (C₂-C₆)alkenyl group, (C₂-C₆)alkynyl group, (C₆-C₁₁)phenyl group or (C₇-C₁₂)benzyl group;
R₃₇ to R₄₀ are respectively (C₁-C₅)alkyl group or (C₁-C₅)haloalkoxy group;
R₄₁ and R₄₂ are respectively H, (C₁-C₅)alkyl group or (C₁-C₅)haloalkoxy group, one of which is H and the other is not H;
R₄₃ and R₄₄ are respectively H, (C₁-C₅)alkyl group or (C₁-C₅)haloalkoxy group.

5. The polypeptide according to claim 1, wherein the substrate is a natural or modified curcuminoid, anthraquinones, chalcone, stilbenoid, coumestan or coumarin.

6. The polypeptide according to claim 5, wherein the curcuminoid is a natural or modified curcumin, bisdemethoxycurcumin, tetrahydrocurcumin or octahydrocurcumin; the anthraquinone is a natural or modified emodin, obtusifolin or aloe-emodin; the chalcone is a natural or modified phloretin, isoliquiritigenin or flavokawain A; the stilbenoid is a natural or modified resveratrol, pterostilbene, piceatannol or oxyresveratrol; the coumestan is a natural or modified coumestrol, wedelolactone or demethylwedelolactone; the coumarin is a natural or modified umbelliferone or 4-hydroxycoumarin.

7. A microorganism comprising a nucleic acid sequence encoding the polypeptide according to claim 1.

8. The microorganism according to claim 7, wherein the nucleic acid sequence is derived from Bacillus subtilis, Bacillus halotolerans, Bacillus atrophaeus, Bacillus mojavensis, Bacillus velezensis or Bacillus amyloliquefaciens.

9. A method for synthesizing a phenolic phytochemical phosphate derivative, comprising: exposing a phenolic phytochemical to the polypeptide according to claim 1 or to a microorganism comprising a nucleic acid sequence encoding the polypeptide according to claim 1 for converting the polyphenol phytochemical to its phosphate derivatives.

10. The method according to claim 9, wherein the phenolic phytochemical is a natural or modified curcuminoid, anthraquinone, chalcone, stilbenoid, coumestan or coumarin.


SEQUENCE LISTING (14 sequences)

SEQ ID NO: 1 - protein, 449 aa, Bacillus subtilis
SFMITDTDMNDFWLNMESNIEGPVSPLFSSFIVPALEYGLKKSMQKFPIGVVVDEVKLYRGHIYSKNQGGQQPPSEDCGKELFPILSEHMYDIINHTYLPFYRTLDQLAQTEHTAESALDAFQKLKAFYLTAYEEHFNIVFPQILLTNKLQAMYQDIQGESENAHFYEMLTGKMNKSLETDRCLWLFSMEVQENPNLLTIFENNKPEQLQEKLEQTDEGRHFLKNVHEFLQEYGWRSVKSHDLIEQIWVENPYFALANIQNYVRNGYHFDNEFQKTKEKREKLYNEFLENIEDPGLRTEFDRYYQWTLNSANIKDDHHFYIDAMLDAKARIFLLKIGELLAENGVIQDREDLWFLYDDEVEQALLHPVSLQEKAEKRRQIFHEYELAQAPAYLGTPTKEQLKAAEEIVGAVIEDEKNTENHIFGIAASSGIATGPVKIIRDANEFSQFA

SEQ ID NO: 2 - protein, 289 aa, Bacillus subtilis
AGAKGMNLIKLTKHGLPVPDGFIIQTNALARFMEDNQLQETSENVESGIISGTFSDELKDELTSSFYKLRESYRSVAVRSSSASEDLEGASFAGQYETYLNIKTEEEFLAKVKECWASFFSGRVSSYKKKMNNQIAEPLMGIVVQGLIDSEMSGVIFSRNPVTHDDRELLISASYGLGEAVVSGSVTPDTFIVNKSSFEIQKEIGAKEIYMESAAEGIAEKETSEDMRSRFCLTDEQVIELAEITKKTEDLYGYPVDIEFGIADHQIYLLQARPITTIDQDKKAAEEKR

SEQ ID NO: 3 - protein, 70 aa, Bacillus subtilis
PGDVLVCKMTTPLWTSLFQDAKAIITDTGGILSHAAIIAREYGIPAVLGTRTATERLRDGDIITVDGSSG

SEQ ID NO: 4 - DNA, 1347 bp, Bacillus subtilis
agcttcatgattaccgacactgatatgaatgatttctggcttaacatggagtctaatattgaaggtccggtgagtccgttattttcatccttcatcgtgccggcattggaatatggcttgaagaagagcatgcaaaagtttccgattggtgtagttgttgatgaagtaaaactttatcgcggacatatttattccaaaaaccaaggtggacagcagcctccttctgaagactgcggcaaagagcttttcccgattttatcggagcatatgtatgacatcatcaatcacacatacctccctttttaccggacactggaccagctcgcacaaactgagcataccgcagaaagcgcactggatgcttttcaaaaactaaaggccttttatctcacggcttatgaagagcacttcaatatcgttttcccgcaaatccttttaacaaacaaactgcaagcgatgtatcaggacattcaaggagagtccgaaaacgctcatttttatgagatgctgacaggaaaaatgaacaaatcactggaaacggaccgttgcttatggctattttctatggaagttcaggagaacccgaaccttctgaccatttttgaaaacaacaagcctgaacagctccaggagaaattagaacaaacagatgaggggagacacttcctgaagaacgtccatgaattcttgcaagaatacggatggagatctgttaaaagtcatgatctgattgaacaaatctgggtggaaaatccgtatttcgctctggctaatattcaaaattatgtccgtaatggctatcattttgacaatgaatttcagaaaacgaaagaaaaacgagagaaattatacaatgaattcttggaaaacatagaagatcccggtttgcgcaccgaatttgaccgctattatcaatggacactgaactctgcaaatataaaagatgatcaccacttttatattgacgccatgctggatgccaaggcgagaatctttctgctgaagataggtgaattgctggcggaaaacggtgtcattcaagatcgtgaggacctttggtttttatatgacgacgaagtggaacaagcgcttcttcaccctgtatccctgcaagaaaaggctgaaaaacgcagacagatttttcatgagtatgagctggcccaagcccctgcctacctcggcaccccgacaaaagaacagctcaaagcagctgaagaaattgtcggcgctgtgatagaggatgaaaaaaacacagagaatcatatttttggcattgcggcatcaagcggcattgcgacaggtccggtgaaaatcattcgggacgccaatgaattttctcaattcgcg

SEQ ID NO: 5 - DNA, 936 bp, Bacillus subtilis
atgaagaaaagaggggtttcaaatatgtattctgttttatttcgccaggcagaagagtccagccagctggctggagcaaaaggaatgaatttgattaaattgaccaaacacggtcttcctgttccggacgggtttattattcaaacgaatgcgctcgcacgttttatggaggacaaccagcttcaagagactagtgaaaacgtcgaaagcgggatcatttctggaacattttcggatgagctgaaagatgagctgactagttccttttataagcttagagaatcatatcgatccgtagccgtgcgttcttcgtctgcttcggaagatttagaaggcgcctcattcgcgggtcaatatgaaacctacttaaatatcaaaacagaggaagagtttctggctaaagtgaaagaatgctgggcctcatttttttctgggcgggtcagcagctataagaaaaaaatgaacaatcaaatcgcagagccgttaatgggaatagtcgttcaggggctgatcgattcagaaatgtcaggtgttatcttcagccgcaaccctgttacccatgatgatagagagcttttaatcagcgccagctacgggttgggtgaagctgttgtttcaggaagtgttaccccagacacgttcattgttaataaatcttcgtttgagattcagaaagaaataggtgcaaaggaaatctacatggagtctgcggcagaaggaattgctgaaaaagaaacgagtgaagacatgcgcagccgtttttgccttacagatgaacaagtgattgaattggctgaaatcacaaaaaaaaccgaagacctgtacggatatcctgtcgatatagaatttggaattgctgatcatcaaatataccttctgcaagctcgcccgattacaaccattgatcaggacaaaaaggcggcagaagaaaaacgc

SEQ ID NO: 6 - DNA, 237 bp, Bacillus subtilis
cctggggacgtactcgtttgcaagatgaccacaccgctatggaccagcctgtttcaagacgccaaagcgataattacagacacaggcggcattttgtctcacgctgcgattattgcccgtgaatacggcattccagccgttctcggcacacgcacggcaaccgaaagactgcgagacggtgacatcatcactgttgacggtagcagcggcaaaatcacagttgtcagccggtcctga

SEQ ID NO: 7 - protein, 831 aa, Bacillus subtilis
MYSVLFRQAEESSQLAGAKGMNLIKLTKHGLPVPDGFIIQTNALARFMEDNQLQETSENVEGGIISGTFSDELKDELTSSFYKLRESYRSVAVRSSSASEDLEGASFAGQYETYLNIKTEEEFLAKVKECWASFFSGRVSSYKKKMNNQIAEPLMGIVVQGLIDSEMSGVIFSRNPVTHDDRELLISASYGLGEAVVSGNVTPDTFIVNKSSFEIQKEIGAKEIYMESAAEGIAEKETSEDMRSRFCLTDEQVIELAEITKKTEDLYGYPVDIEFGIADHQIYLLQARPITTIDQDKKAAEEKRSFMITDTDMNDFWLNMESNIEGPVSPLFSSFIVPALEYGLKKSMQKFPIGVVVDEVKLYRGHIYSKNQGGQQPPSEDCGKELFPILSEHMYDIINHTYLPFYRTLDQLAQTEHTAESALEAFQKLKAFYLTAYEEHFNIVFPQILLTNKLQAMYQDIQGESENAHFYEMLTGKMNKSLETDRCLWLFSVEVQENPNLLAIFENNKPEQLQEKLEQTDEGRHFLKNVHEFLQEYGWRSVKSHDLIEQIWVENPYFALANIQNYVRNGYHFDNEFQKTKEKREKLYNEFLESIEDPGLRTEFDRYYQWTLNSANIKDDHHFYIDAMLDAKARIFLLKIGELLAENGVIQDREDLWFLYDDEVEQALLHPVSLQEKAEKRRQIFHEYELAQAPAYLGTPTKEQLKAAEEIVGAVIEDEKNTENHIFGIAASSGIATGPVKIIRDANEFSQFAPGDVLVCKMTTPLWTSLFQDAKAIITDTGGILSHAAIIAREYGIPAVLGTRTATERLRDGDIITVDGSSGKITVVSRS

SEQ ID NO: 8 - protein, 831 aa, Bacillus halotolerans
MYSVLFRQAEESSQLAGAKGMNLIKLTKHGLPVPDGFIIQTNALARFMEDNQLHDSSENIENAIIAGTFSDELKNELTSSFYELRESYRSVAVRSSSASEDLEGASFAGQYETYLNIKTEEEFLAKVKECWASFFSGRVSRYKKKMNNQIAEPLMGVVVQGLINSEISGVIFSQNPVTHDDRELLISASYGLGEAVVSGSVTPDTYIVHKASFEIQKEMGLKEIYMESAAEGIAEKETSEDMRSRFCLTDEQVIELAEITKKTEALYGYPVDLEFGIADHKLYLLQARPITTIEQDKKAAEEERNFMITDEDMDDFWLNMESNIEGPVSPLFSSFIVPALEYGLKKSMQQFPIGVIVDEVKVYRGHIYSKNQGGQQQPPEDSAEELFPILSERMYDIIHNTYLPFYRTLDQLAQTEHTPESALDAFKKLKAFYLTAYEEHFNIVFPQILLTNKLQAMYQNIQGETENSHFYEMLTGVMNKSLETDRRLWQFSVEVRENPNLTAIFEHTQPQQLQKTLEQADEGRRFLQKVNAFLQEYGWRSVKSHDLIEQTWAENPYYALTHIQNYVRNGYHFDNEFKKTIEKREKLYNEFLQSIEDPVLQKEFERYYQWTLNSSNIKDDHHFYIDAMLDAKARVFLLKVGELLAKSGVIRDREDLWFLYDDEVENALLHPVSLQEKAEKRRQIFHEYELAQAPAYLGTPTKAQLKAAEEIVGAVIEDEKNTENDIFGIAASSGIATGPVKLIRDASEFSRFAPGDILVCKMTTPLWTSLFQDAKAIITDTGGILSHAAIIAREYGIPAVLGTRAATERLRDGDIVTVDGNSGKITVVSRS

SEQ ID NO: 9 - protein, 831 aa, Bacillus mojavensis
MYSVLFRQAEESSQLAGAKGMNLIKLTKHGLPVPDGFIIQTNALARFMEDNQLHDSSENIENAIIAGTFSDELKNELTSSFYELRESYRSVAVRSSSASEDLEGASFAGQYETYLNIKTEEEFLAKVKECWASFFSGRVSRYKKKMNNQIAEPLMGVVVQGLINSEISGVIFSQNPVTHDDKELLISASYGLGEAVVSGSVTPDTYIVHKASFQIQKEMGLKEIYMESAAEGIAEKETSEDMRSRFCLTDEQVIELAEITKKTEALYGYPVDLEFGIANHKLYLLQARPITTIEQDKKAAEEERNFMITDEDMDDFWLNMESNIEGPVSPLFSSFIVPALEYGLKKSMQQFPIGVIVDEVKVYRGHIYSKNQGGQQQPPEDSAEELFPILAERMYDIIHNTYLPFYRTLDQLAQTEHTPESALEAFKKLKAFYLTAYEEHFNIVFPQILLTNKLQAMYQNIQGEAENSHFYEMLTGVMNKSLETDRRLWQFSVEARENPNLTAIFEHTEPQQLQKTLEQIDEGRCFLQKVNEFLQEYGWRSVKSHDLIEQTWAENPYYALTHIQNYVRNGYHFDNEFKKTIEKREKLYNEFLQSIEDPVLQKEFERYYQWTLNSSNIKDDHHFYIDAMLDAKARVFLLKVGELLAKSGVIRDREDLWFLYDDEVENALLHPVSLQEKAEKRRQIFHEYELAQAPAYLGTPTKAQLKAAEEIVGAVIEDEKNTENDIFGIAASSGIATGPVKLIRDASEFSRFAPGDILVCKMTTPLWTSLFQDAKAIITDTGGILSHAAIIAREYGIPAVLGTRAATERLRDGDIVTVDGNSGKITVVSRS

SEQ ID NO: 10 - protein, 834 aa, Bacillus atrophaeus
MYSVLFNKAEESSQLAGAKGMNLIKMTKNGLPVPDGFIIQTNALARFMEDNQLHETKEDIENNIINGIFSDELKNELTASFYELRESYASVAVRSSSASEDLEGASFAGQYETYLNVKTEDEFLGKVKECWASFFSARVGRYKEKMKNNHGKPLMGVVVQGLIESEVSGVIFSRNPVTHDDGELLISASYGLGEAIVSGSVTPDTFIVNKDSFAIQKEMGLKELYMVSQSEGIAERETNEEMRNRYCLNDEHIRELAALTKKTEALYGFPVDLEFGIAENTLYLLQARPITTIDQDKEAAEEERDFMITKEDMNDFWLNMESNIEGPISPLFSSLIVPAMEHGLKKRSEQFPIGVTIEEVKQYRGHIYSKQKGGDPTEAAKAAEAAEELFPHLAERMYGILNKTFLPFYETLDELSAASHTPESALNAFKKLKAFYMEAYDEHFNIVFPQLLLNTKLETMYQQVQGDTENSHFHEMLTGKMNKSLETDRHLWLLSNEVKKNAALKQVFETHQAEELQETLAQTSDGKLFLDKVNEFLREYGWRSVKSHDLIEQIWAENPYYALSHIQNYVRNGYHFDNEFNKTIEKRKQLYNEFLQQIEDEAFRKEYDRYYQWMLNSSVIRDDHHFYIDAMLDAKARIFLLRIGEMLADSGVIDDKEDLWYLYDDEIENALLHPVPLQAKTAKRREVFKEYELVHAPSYLGSPTAEQLKAAEDIVGSVTEDEKNTEDHIYGVAASSGIVSGPVKVIRDANEFSRFSPGDVLVCKMTTPLWTSLFQDAKAVITDTGGILSHAAIIAREYGLPAVLGTRAATDRLRDGDVVTVDGTSGKITIVSRA

SEQ ID NO: 11 - protein, 833 aa, Bacillus velezensis
MYSVRFQKAEESSRSAGAKGMNLIKLTKNGLPVPGGFIIQTNALTRFMEDNGLQADQEQLERAIMNGTFSKELREELTASFYELRESYASVAVRSSSASEDLEGASFAGQYETYLNIKTEEEFLGKVKECWASFFSARVSGYKEKMNNDTAEPLMGVVVQGLINSEVSGVIFSRNPVTHDDGELMISASYGLGEAIVSGSVTPDTFIVNKDTFQIEKEIGLKEMYIVSKDEGVTEKETSADMRSRFCLDDESVKELAMLTIKTEELYGYPVDLEFGFAENKLYLLQARPITTIAQDQKAADEEREFIMTPRDQKDFWLNMEANIEGPVSPLFASLIVPALEYGLKESTKAFPVMGIEIERVKLHQGHVFSRQHKTDDKLPAEQLEALFPVLADRMYDIIQETFLPFYQKLDELAHTDHTPETALDAFRNLQDFYLKGYEEHFNIVLPQVALNMILESMYGQVEKENTSLLYEMLAGVMNKSLETDRQLWLLSGQVKDSPELRRVFTVSHADELHQTLLQSNEGKRFLEQVGEFLQEYGWRSVKSHDLIEQTWAENPYFALANIQNYVRNGYDFDSEFHKTIEKRKQLYADFMDRIEDDGFRETFDRYYQWTLSSSVIKDDHHFYIDAMLDAKARLCLLKIGELLQKQGVIDDREDMWYLYSDEVEKALASPVPMQEKAAERKQLFQQYQLLEAPAYLGTPTSEQLKTAEQITGSITEDEKNTEHQIYGLAASSGIASGPVKVIRDASEFSRFSTGDVLVCKMTTPLWTSLFQDAKAVITDTGGILSHAAIIAREYGLPAVLGTRAATDRLNDGDIVTVDGTNGKITIVKRASC

SEQ ID NO: 12 - protein, 833 aa, Bacillus amyloliquefaciens
MYSVRFQKAEESSRSAGAKGMNLIKLTKNGLPVPDGFIIQTNALTRFMEDNGLQADQEQMERAIMNGTFSKELREELTASFYELRETYASVAVRSSSASEDLEGASFAGQYETYLNIKTEEEFLGKVKECWASFFSARVSGYKEKMNNDTAEPLMGVVVQGLINSEVSGVIFSRNPVTHDDGELMISASYGLGEAIVSGSVTPDTFIVNKDTFQIEKEIGLKEMYIVSKDEGVTEKETTADMRNRFCLDDESVKELAMLTIKTEELYGYPVDLEFGFAENKLYLLQARPITTIVQDQKAADEEREFIMTPRDQKDFWLNMEANIEGPVSPLFASLIVPALEYGLKESTKAFPVMGIEIERVKLHQGRVFSRQHKTDDKLPAEQLEALFPVLADRMYDIIQETFLPFYQKLDELAHTDHTPETALDAFRNLQDFYLKGYAEHFNIVFPQVALNMILESMYGQVEKENTSHLYEMLAGVMNKSLETDRQLWLLSGQVKDNPELRRVFTVSHADELHQTLLQSNEGKRFLEQVGEFLQEYGWRSVKSHDLIEETWAENPYFALANIQNYVRNGYDFDSEFHKTIEKRKQLYADFMDRIEDDGFRETFDRYYQWTLSSSVIKDDHHFYIDAMLDAKARLCLLKIGELLQKQGVIDDREDMWYLYSDEVEKALVSPVPMQEKAAERKQLFQQYQLLEAPAYLGTPTSEQLKTAEQITGSITEDEKNTEHQIYGLAASSGIASGPVKVIRDASEFSRFSTGDVLVCKMTTPLWTSLFQDAKAVITDTGGILSHAAIIAREYGLPAVLGTRAATDRLNDGDIVTVDGTNGKITIVKRASC

SEQ ID NO: 13 - protein, 839 aa, Bacillus subtilis
MKKRGVSNMYSVLFRQAEESSQLAGAKGMNLIKLTKHGLPVPDGFIIQTNALARFMEDNQLQETSENVESGIISGTFSDELKDELTSSFYKLRESYRSVAVRSSSASEDLEGASFAGQYETYLNIKTEEEFLAKVKECWASFFSGRVSSYKKKMNNQIAEPLMGIVVQGLIDSEMSGVIFSRNPVTHDDRELLISASYGLGEAVVSGSVTPDTFIVNKSSFEIQKEIGAKEIYMESAAEGIAEKETSEDMRSRFCLTDEQVIELAEITKKTEDLYGYPVDIEFGIADHQIYLLQARPITTIDQDKKAAEEKRSFMITDTDMNDFWLNMESNIEGPVSPLFSSFIVPALEYGLKKSMQKFPIGVVVDEVKLYRGHIYSKNQGGQQPPSEDCGKELFPILSEHMYDIINHTYLPFYRTLDQLAQTEHTAESALDAFQKLKAFYLTAYEEHFNIVFPQILLTNKLQAMYQDIQGESENAHFYEMLTGKMNKSLETDRCLWLFSMEVQENPNLLTIFENNKPEQLQEKLEQTDEGRHFLKNVHEFLQEYGWRSVKSHDLIEQIWVENPYFALANIQNYVRNGYHFDNEFQKTKEKREKLYNEFLENIEDPGLRTEFDRYYQWTLNSANIKDDHHFYIDAMLDAKARIFLLKIGELLAENGVIQDREDLWFLYDDEVEQALLHPVSLQEKAEKRRQIFHEYELAQAPAYLGTPTKEQLKAAEEIVGAVIEDEKNTENHIFGIAASSGIATGPVKIIRDANEFSQFAPGDVLVCKMTTPLWTSLFQDAKAIITDTGGILSHAAIIAREYGIPAVLGTRTATERLRDGDIITVDGSSGKITVVSRS

SEQ ID NO: 14 - protein, 16 aa, artificial
  conserved motif
DDHHFYIDAMLDAKAR